SEQUENCE LISTING (41 sequences)

SEQ ID NO: 1 - DNA, 1213 bp, Artificial Sequence
  Codon-optimized synthetic Cannabis sativa
atcaataataatcttcatgggattgtctttggtttgtactttttcttttcaaaccaattatcatacccttttgaatccacacaacaaaaatccaaaaaattctcttttgagttatcaacatccaaaaactccaattattaaaagttcttatgataatttcccatctaaatactgtcttactaaaaatttccatttgttgggtcttaatagtcataatagaatctcttctcagtctaggagtattagagccggttctgaccaaatcgagggatctccacaccacgagtctgacaacagtatcgccaccaagatcttgaacttcggtcacacttgttggaagcttcagaggccatacgtcgtcaagggtatgatctctattgcttgtggtcttttcggtagggagcttttcaacaatagacacttgttctcttggggtcttatgtggaaggccttcttcgcccttgtcccaatcctttctttcaacttttttgctgctattatgaatcaaatttatgacgtcgacatcgataggatcaacaagccagacttgcctcttgtctctggagagatgtctatcgagaccgcttggatcttgagtatcatcgtcgcccttaccggtttgattgttaccatcaaattgaaaagtgctcctctttttgtttttatttacatcttcggaatctttgccggtttcgcctactctgtcccacctatcagatggaagcagtacccattcactaattttttgatcaccattagttctcacgtcggacttgccttcacctcttactctgccactacctctgccttgggtttgccattcgtctggagaccagccttttctttcatcatcgccttcatgaccgtcatgggaatgaccatcgccttcgccaaagacatctctgacatcgagggtgatgccaagtacggtgtttctaccgtcgccaccaagttgggtgctaggaacatgaccttcgtcgtctctggagtccttcttttgaattaccttgtctctatctctatcggaatcatctggccacaagttttcaaatctaatattatgatcctttctcatgccatcttggccttttgtttgatcttccagactagagagttggccttggccaactacgccagtgccccatctagacaattcttcgaattcatttggcttctttattatgccgaatattttgtttatgtctttatttaa

SEQ ID NO: 2 - DNA, 1638 bp, Artificial Sequence
  Codon-optimized synthetic Cannabis sativa
atgatttttgatggtactactatgtctattgctattggtttgttgtctactttgggtattggtgctgaagctatttccatagctaacccacaagaaaatttcttaaaatgtttttctgagtatattccaaataatcctgctaatccaaagtttatttatactcaacatgatcaattatatatgagtgtgctgaactctactattcaaaatctgagatttacttctgatactactccaaaaccattggttattgttactccatctaatgtttctcatattcaagcttctattttgtgttctaaaaaagttggtttgcaaattagaactagatctggtggtcatgatgctgaaggtttgtcttatatttctcaagttccatttgctattgttgatttgagaaatatgcatactgttaaagttgatattcattctcaaactgcttgggttgaagctggtgctactttgggtgaagtttattattggattaatgaaatgaatgaaaatttttcttttcctggtggttattgtccaactgtgggtgttggtggtcatttttctggtggtggttatggtgctttgatgagaaattatggtttggctgctgataatattattgatgctcatttggttaatgttgatggtaaagttttggatagaaaatctatgggtgaagatttgttttgggctattagaggtggtggtggtgaaaattttggtattattgctgcttgcaaaataaaattggttgttgtaccctccaaagctactatcttttctgttaagaaaaatatggaaattcatggtttggttaaattgttcaataaatggcagaatattgcttataaatatgataaggacttgatgttgactacccattttaggactagaaacattactgataatcatggtaagaacaaaactactgttcatggttatttttcttctatttttttgggtggtgttgattctttggttgatttgatgaataaaagcttccctgaattgggtattaaaaaaactgattgtaaagaactgtcttggattgatactacaattttttattctggtgttgttaattataacactgctaactttaagaaagagattttgttagatagatctgctgggaaaaaaactgctttttctataaaattggattatgtgaaaaaactaattcctgaaactgcaatggttaaaatccttgaaaaattgtatgaagaagaagttggtgttggtatgtatgttttgtatccatatggtggtattatggatgaaatttctgaatctgctattccatttccacatagagctggtattatgtatgaattgtggtacacagctacttgggaaaaacaagaagacaatgagaaacatattaactgggttagatctgtttataattttactactccatatgtttctcaaaatccaagattggcttatttgaattatagagatttggatttgggtaaaactaatcctgaatctccaaataactatacacaagctagaatttggggtgaaaaatattttggtaagaacttcaatagattggttaaggttaaaactaaggctgatccaaataatttttttagaaatgaacaatctattccaccattgccaccaagacatcattaa

SEQ ID NO: 3 - DNA, 1185 bp, Artificial Sequence
  Codon-optimized synthetic Ralstonia eutropha
atgactagggaggtcgttgttgtctctggtgttagaaccgccatcggtaccttcggtggtagtcttaaggacgtcgccccagccgaattgggtgctcttgtcgttagagaagcccttgctagagcccaagtctctggagatgatgtcggacacgtcgtcttcggtaacgtcatccaaaccgagccaagggatatgtaccttggtagagtcgctgccgtcaatggtggtgttaccatcaacgctccagctcttaccgtcaacagactttgcggttctggacttcaagctatcgtctctgctgcccaaaccatcttgttgggagacaccgatgtcgctatcggaggtggtgccgagagtatgtctagagccccatatttggccccagctgctagatggggtgctagaatgggtgatgccggtcttgtcgacatgatgcttggagcccttcacgacccattccatagaattcacatgggagtcaccgctgagaacgtcgccaaggaatacgacatcagtagggcccagcaagacgaggccgctttggagagtcacagaagagcttctgctgccatcaaggccggatacttcaaggaccagatcgtcccagttgtcagtaagggtaggaagggtgacgtcactttcgacaccgatgagcatgttaggcatgacgccaccatcgatgacatgaccaagttgaggccagttttcgtcaaggaaaacggtaccgtcaccgccggtaatgccagtggtttgaatgatgccgctgccgctgttgtcatgatggaaagagccgaggctgaaagaaggggtttgaagccattggctagacttgtctcttacggtcatgccggtgttgatccaaaggctatgggaatcggaccagttccagctaccaaaatcgccttggagagggccggtcttcaagtcagtgacttggacgtcatcgaggctaatgaagccttcgccgctcaagcttgcgctgttaccaaggccttgggtcttgatccagccaaggtcaacccaaacggtagtggtatctctcttggacaccctatcggagctaccggtgctttgatcaccgtcaaggcccttcacgagcttaatagagtccaaggtaggtacgcccttgtcactatgtgtatcggtggtggacaaggtatcgccgccatctttgagaggatctaa

SEQ ID NO: 4 - DNA, 855 bp, Artificial Sequence
  Codon-optimized synthetic Cupriavidus necator
atgagtatcagaaccgtcggtatcgtcggagctggtaccatgggaaatggaatcgcccaagcttgtgccgtcgttggattgaacgtcgtcatggtcgacatcagtgatgccgctgttcaaaagggtgtcgctaccgtcgctggttctttggatagattgatcaagaaggagaagttgaccgaggccgacaaagcctctgctttggctagaatcaagggatctaccagttacgacgaccttaaggccaccgacatcgttatcgaagctgccaccgagaactacgacttgaaggtcaagattttgaaacaaattgattctatcgttggagaaaatgttattattgcttctaatacctcttctatcagtatcaccaagcttgctgccgtcacttctagagccgatagattcatcggtatgcacttcttcaacccagtcccagttatggcccttgtcgagttgattagaggtttgcaaaccagtgataccacccatgccgctgtcgaggccctttctaagcagcttggaaagtaccctatcaccgttaggaactctcccggtttcgtcgtcaatagaattttgtgtcctatgatcaacgaggctttctgcgtccttggtgaaggacttgctagtccagaggagatcgacgagggaatgaagttgggatgcaaccacccaatcggacctttggctcttgccgacatgatcggattggacaccatgcttgccgtcatggaggtcctttacaccgagttcgctgaccctaagtatagaccagccatgttgcttagggagatggtcgccgccggttaccttggtaggaagaccggtaggggtgtctacgtttattctaaataa

SEQ ID NO: 5 - DNA, 786 bp, Artificial Sequence
  Codon-optimized synthetic Clostridium acetobutylicum
atggagttgaataacgttatccttgagaaggagggaaaggtcgctgttgtcaccatcaatagacctaaggccttgaacgcccttaactctgacaccttgaaggagatggattatgttattggagagatcgaaaacgactctgaggtccttgccgttatcttgaccggtgccggtgagaagagtttcgtcgccggtgccgatatctctgaaatgaaggaaatgaacactatcgaaggaagaaaattcggaatcttgggtaacaaggtctttagaaggcttgagttgttggagaagccagttatcgctgccgttaatggtttcgctcttggtggtggttgcgagattgccatgagttgcgacatcagaatcgccagtagtaacgctagattcggtcagccagaagtcggtttgggtatcacccccggtttcggaggtacccagaggcttagtaggcttgtcggtatgggaatggccaagcagcttatcttcaccgcccagaacatcaaggccgatgaggcccttaggatcggattggtcaacaaggtcgtcgagccatctgaacttatgaacaccgccaaggagatcgccaacaagatcgtcagtaacgccccagtcgccgttaagttgagtaagcaagctatcaatagaggaatgcagtgcgacatcgataccgctttggccttcgaatctgaggccttcggtgagtgcttcagtaccgaggatcagaaggatgccatgaccgccttcatcgaaaagaggaagattgaaggtttcaagaatagataa

SEQ ID NO: 6 - DNA, 1194 bp, Artificial Sequence
  Codon-optimized synthetic Treponema denticola
atgattgttaaaccaatggttaggaataacatctgcttgaacgcccaccctcaaggttgtaagaagggagtcgaggaccagattgagtacaccaagaagagaatcactgccgaggtcaaggctggtgccaaagcccctaagaacgtcttggtcttgggatgctctaacggatacggtttggcctctaggatcaccgccgctttcggttatggagctgccaccatcggtgtctctttcgagaaggccggttctgaaaccaagtacggtacccccggttggtacaacaaccttgcctttgacgaggccgccaaaagggaaggtctttacagtgtcaccatcgacggagatgccttctctgacgagatcaaggcccaagttatcgaagaagccaaaaagaagggaatcaaatttgacttgatcgtctacagtcttgcctctccagttagaaccgatccagataccggaatcatgcacaaatctgttcttaaaccttttggaaagaccttcactggtaagaccgttgacccattcaccggagagcttaaggagatctctgccgaaccagctaacgacgaagaagccgccgccaccgtcaaggttatgggaggtgaggactgggagaggtggatcaagcagttgtctaaggagggtcttcttgaggagggttgcatcaccttggcctacagttacatcggaccagaggccacccaagccttgtatagaaagggtaccatcggtaaggccaaggagcacttggaagctaccgcccacagattgaacaaggagaacccaagtatcagagccttcgtcagtgtcaacaagggtcttgtcactagagcctctgccgtcatcccagtcatccctctttatttggcctctcttttcaaggtcatgaaggagaaaggaaaccacgagggatgcattgagcagatcactagactttacgccgagaggctttataggaaggacggtaccatcccagtcgacgaggaaaatagaattagaatcgatgactgggaacttgaggaggacgtccagaaagccgtttctgccttgatggagaaggtcaccggtgaaaatgccgagagtttgaccgacttggccggttacagacacgacttcttggccagtaacggttttgacgttgagggaatcaactacgaggccgaagttgagaggttcgacagaatctaa

SEQ ID NO: 7 - DNA, 1158 bp, Artificial Sequence
  Codon-optimized synthetic Cannabis sativa
atgaatcatttgagggccgagggaccagctagtgttttggccatcggtaccgccaacccagaaaacatccttttgcaagatgaatttccagattattatttcagagttaccaagagtgaacatatgactcaattgaaggaaaagtttagaaaaatttgtgataagtctatgattaggaaaaggaattgctttttgaacgaagagcaccttaagcaaaaccctaggcttgtcgagcacgaaatgcagaccttggacgccagacaagacatgttggtcgtcgaggtccctaagcttggaaaggatgcttgtgccaaagctatcaaggagtggggacagccaaagagtaagatcactcatttgatctttacctctgcttctaccaccgatatgcccggtgctgactatcactgcgccaagttgttgggtttgagtcctagtgtcaagagggtcatgatgtaccagcttggttgctacggtggaggaaccgtcttgaggatcgccaaggacatcgctgagaacaacaaaggtgctagagtcttggccgtttgctgtgacatcatggcttgtttgtttagaggtccttctgagtctgaccttgagcttttggtcggacaagccatctttggtgacggtgctgctgccgttattgttggtgctgaaccagatgagagtgtcggtgagaggccaatcttcgaacttgtctctaccggtcagaccatcttgcctaactctgagggtaccatcggaggtcacattagagaagccggtttgatcttcgatcttcataaggatgttccaatgcttattagtaataatatcgagaaatgcttgatcgaggccttcaccccaatcggaatttctgactggaacagtatcttctggatcacccaccccggtggtaaggccattttggacaaggtcgaggagaagcttcaccttaagagtgacaagttcgtcgactctaggcacgtcttgtctgagcacggtaacatgtcttctagtaccgttttgttcgtcatggacgagttgaggaagaggagtttggaggagggtaaatctaccaccggtgacggattcgagtggggagtccttttcggtttcggtcccggtttgaccgtcgagagggtcgtcgtcagatctgtccctatcaaatattaa

SEQ ID NO: 8 - DNA, 306 bp, Artificial Sequence
  Codon-optimized synthetic Cannabis sativa
atggctgttaaacatttgatcgttttgaaattcaaggacgagatcaccgaagcccagaaggaggagttcttcaagacctacgtcaaccttgtcaacatcatcccagccatgaaggacgtctactggggtaaagacgtcacccagaagaacaaggaggagggttacacccacatcgttgaagtcaccttcgagtctgtcgagaccatccaagattacatcatccacccagcccatgtcggtttcggtgacgtctatagaagtttctgggagaagcttcttatttttgattataccccaagaaaataa

SEQ ID NO: 9 - DNA, 2163 bp, Artificial Sequence
  Codon-optimized synthetic Cannabis sativa
atgggaaagaactataaaagtcttgacagtgtcgtcgcctctgacttcattgcccttggtatcaccagtgaggtcgctgaaacccttcacggtagacttgccgagattgtctgcaactacggagccgctacccctcagacttggattaacatcgccaaccatattttgagtccagacttgcctttcagtttgcaccaaatgcttttttatggttgttacaaggatttcggtccagcccctccagcttggattccagacccagagaaagtcaagagtaccaacttgggagcccttcttgaaaaaagaggtaaagaattcttgggagttaagtataaagatccaatcagtagtttcagtcatttccaagaattttctgttagaaacccagaggtttactggaggaccgtccttatggacgagatgaagatctcttttagtaaagatccagaatgtattttgagaagagatgacatcaacaaccccggtggatctgagtggttgcccggtggataccttaactctgctaaaaattgcttgaatgttaattctaataaaaaattgaatgataccatgatcgtctggagggacgagggaaacgacgacttgccattgaacaagttgaccttggatcagcttaggaagagggtctggttggttggttacgcccttgaggagatgggtttggagaaaggttgcgccatcgccatcgacatgcctatgcacgttgacgccgttgttatttatcttgccatcgtcttggctggttacgtcgttgtcagtatcgccgacagtttttctgccccagaaatcagtactagattgagattgtctaaggccaaagccattttcacccaagatcatatcattagaggaaagaaaagaattccactttactctagggtcgttgaggccaagagtcctatggccatcgtcatcccttgtagtggtagtaacatcggagccgaattgagagacggtgacatctcttgggactacttcttggaaagggccaaagaattcaaaaattgtgagttcaccgctagagagcagccagttgatgcttacaccaatatcttgttctcttctggaaccaccggtgaaccaaaggccattccatggactcaagctacccctcttaaggccgccgctgacggatggagtcacttggatattagaaagggagacgtcatcgtctggcctaccaatttgggttggatgatgggtccatggttggtctacgcctctcttcttaacggagcctctatcgccttgtacaacggtagtccacttgttagtggtttcgccaagttcgtccaagacgctaaggtcaccatgcttggagtcgtcccatctatcgtcagatcttggaaatctactaactgcgtctctggttacgactggtctaccattagatgcttcagttctagtggtgaagccagtaacgtcgacgagtacctttggcttatgggaagggccaattacaagccagttattgagatgtgcggtggaaccgaaatcggtggagccttctctgctggttctttcttgcaagcccagtctttgagttctttctcttctcagtgcatgggttgcaccttgtatatccttgataagaacggatacccaatgccaaagaacaagcccggtatcggtgagcttgctttgggtccagttatgttcggagcctctaaaacccttttgaacggtaaccatcatgacgtctatttcaagggtatgccaaccttgaacggtgaagtcttgaggaggcacggtgatatttttgagcttacctctaatggttactaccacgcccacggaagagctgacgataccatgaacatcggaggtattaagatctcttctatcgagatcgagagggtctgcaacgaagtcgacgacagagtcttcgaaaccaccgctatcggagttcctccattgggtggaggtccagagcagcttgtcatcttctttgttttgaaggactctaacgatactactatcgatcttaatcaacttagactttcttttaatcttggacttcagaaaaaattgaacccattgttcaaagtcactagagtcgtcccattgagttctcttccaaggaccgccaccaacaagatcatgaggagggtcttgaggcaacagttcagtcacttcgaataa

SEQ ID NO: 10 - DNA, 6702 bp, Saccharomyces cerevisiae
atgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactagctgatggtggtcttttgattgccataggcggtaaatcgcataccatctattggaaagaagaagttgctgctacaagattatccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgttgctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaatggaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattattttaactggtgctcctgcaatcaacaaaatgctgggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaataa

SEQ ID NO: 11 - DNA, 1584 bp, Saccharomyces cerevisiae
atggctgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 12 - DNA, 2412 bp, Artificial Sequence
  Codon-optimized synthetic Enterococcus faecalis
atgaaaaccgtcgtcatcatcgatgctcttaggactccaatcggtaaatataagggaagtctttctcaagttagtgccgtcgaccttggtactcacgtcaccacccagttgcttaagaggcactctaccatcagtgaagagatcgaccaagttattttcggtaacgtcttgcaagctggaaatggtcagaacccagctaggcaaatcgccatcaacagtggtttgtctcacgagatcccagctatgactgtcaacgaagtctgcggatctggtatgaaggccgtcatccttgctaagcagcttatccagttgggagaggctgaggtcttgatcgccggtggaatcgagaacatgagtcaagctcctaagcttcagagattcaactacgagaccgagtcttatgacgcccctttcagttctatgatgtacgacggattgaccgacgctttctctggacaagccatgggtcttactgccgagaatgtcgccgagaagtaccatgttactagagaggagcaagaccagttcagtgttcactctcagcttaaggctgcccaagctcaagccgagggaattttcgctgacgaaatcgcccctcttgaagtctctggaacccttgtcgagaaggacgagggaatcagacctaactcttctgtcgaaaagcttggtaccttgaagaccgtcttcaaggaagacggaaccgttaccgccggtaatgccagtaccatcaatgacggtgccagtgctttgatcattgctagtcaagaatatgccgaggcccacggtttgccttacttggccatcattagagactctgtcgaggttggtatcgacccagcttacatgggtatctctcctattaaggctatccaaaaacttttggctagaaaccaacttaccaccgaagagattgatttgtacgagatcaatgaggctttcgctgccaccagtattgtcgtccagagggagttggccttgccagaggagaaggttaacatctacggtggtggtatctctttgggtcatgctatcggtgctaccggtgctagattgttgacctctctttcttaccaattgaaccagaaggagaagaagtatggagtcgccagtctttgcatcggtggtggattgggtcttgctatgcttttggagaggccacagcagaagaaaaacagtagattttatcagatgagtccagaagagagattggcctctcttttgaacgaaggacagatctctgccgacaccaagaaggagtttgagaataccgctttgtctagtcaaatcgctaaccatatgattgagaatcaaatcagtgaaaccgaggttccaatgggtgtcggtttgcatttgactgtcgacgagactgactacttggttccaatggctaccgaggaacctagtgtcatcgctgctctttctaacggtgccaagatcgcccaaggttttaagaccgtcaaccagcaaagattgatgaggggacagatcgtcttttacgacgtcgccgatgccgaatctcttatcgacgaacttcaagttagagagaccgagatcttccaacaagctgagctttcttacccatctatcgtcaagagaggaggaggattgagggaccttcagtatagggccttcgacgagtctttcgtcagtgtcgacttcttggtcgatgtcaaggatgccatgggtgccaacatcgtcaacgccatgttggagggtgtcgccgagttgtttagggagtggttcgctgaacaaaaaattctttttagtatcctttctaactacgccaccgagagtgtcgtcactatgaagaccgctatcccagtttctagacttagtaagggatctaacggaagggagatcgccgaaaagattgtccttgcctctaggtacgcctctttggacccttatagggccgtcacccacaataagggaatcatgaacggtattgaggccgttgtccttgccactggaaacgataccagagctgtctctgcctcttgccatgccttcgccgttaaagagggaaggtaccaaggtcttacctcttggaccttggacggagagcagttgatcggtgagatcagtgtccctttggctcttgctactgtcggtggagccaccaaggttttgccaaagagtcaagccgccgctgaccttttggccgtcaccgacgctaaggagttgagtagggttgtcgccgctgtcggtcttgctcagaaccttgctgctcttagggcccttgtctctgagggaatccagaagggacacatggctcttcaagctaggagtcttgccatgaccgtcggagccactggtaaagaggttgaggctgtcgcccagcaacttaagagacagaaaaccatgaaccaagatagggctttggctatcttgaatgatttgagaaaacaataa

SEQ ID NO: 13 - DNA, 1152 bp, Artificial Sequence
  Codon-optimized synthetic Enterococcus faecalis
atgactattggtattgataaaatttctttcttcgttccaccatactacatcgacatgaccgccttggctgaagccagaaacgtcgaccccggtaagttccacatcggaatcggacaagaccagatggctgtcaaccctatcagtcaagatatcgtcaccttcgctgctaacgctgccgaagccatcttgaccaaggaggataaggaggccattgacatggtcatcgtcggaaccgagagtagtatcgacgagtctaaggccgctgctgtcgtccttcatagattgatgggtatccagcctttcgctagatctttcgagatcaaggaggcttgttacggtgctaccgctggacttcagttggccaagaatcatgtcgcccttcacccagataagaaggttttggtcgttgccgccgatatcgccaagtacggattgaacagtggaggtgagccaacccaaggtgccggtgccgttgccatgttggtcgcctctgaacctagaattttggctcttaaggaggacaacgtcatgcttacccaagatatctacgacttctggagacctaccggtcacccatacccaatggtcgacggaccattgagtaacgagacctacattcagtctttcgcccaagtttgggatgaacacaagaagaggaccggtttggacttcgctgattacgatgccttggccttccatatcccatacaccaaaatgggaaaaaaggcccttcttgccaaaatctctgaccagaccgaggctgagcaagaaaggatcttggctagatacgaagagtctatcatctactctagaagggtcggtaacttgtacaccggttctctttacttgggtttgatctctttgttggagaacgctaccacccttaccgccggaaaccagatcggtttgttcagttacggttctggagccgttgccgaattcttcactggtgagcttgtcgccggttaccagaatcaccttcagaaggagacccatcttgcccttttggacaacagaaccgagcttagtatcgccgagtacgaggccatgttcgctgagactcttgacaccgacatcgaccaaacccttgaggatgaattgaagtattctatctctgctattaacaatactgttagaagttatagaaactaa

SEQ ID NO: 14 - DNA, 1059 bp, Saccharomyces cerevisiae
atggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttactggttggtcgccgatgatatgatggacaagtccattaccagaagaggccaaccatgttggtacaaggttcctgaagttggggaaattgccatctgggacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatag

SEQ ID NO: 15 - DNA, 1332 bp, Saccharomyces cerevisiae
atgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataa

SEQ ID NO: 16 - DNA, 1356 bp, Saccharomyces cerevisiae
atgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataa

SEQ ID NO: 17 - DNA, 1191 bp, Saccharomyces cerevisiae
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 18 - DNA, 867 bp, Saccharomyces cerevisiae
atgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataa

SEQ ID NO: 19 - DNA, 1503 bp, Saccharomyces cerevisiae
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 20 - DNA, 2052 bp, Saccharomyces cerevisiae
atgacaatcaaggaacataaagtagtttatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttattatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatctatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaataa

SEQ ID NO: 21 - DNA, 1047 bp, Saccharomyces cerevisiae
atgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataa

SEQ ID NO: 22 - DNA, 1749 bp, Saccharomyces cerevisiae
atggtacaagaatcacgtccagggagtgtaagaagttactcggtcggttaccaagcaaggtccagatcgagttctcaaagaagacattcgttaacacgccaacgttcctcgcaaagactgattagaaccatcagtatcgagtctgatgtgtctaatattactgacgatgacgatttgagagctgtcaatgagggagtagcgggtgtgcaactggacgtctctgaaaccgcaaataagggaccaagaagagcatcagcaactgatgtcacagatagtttgggttcgacttcgtcggaatatattgagattccctttgttaaggaaacattggatgcaagtttaccttcggattatctgaagcaggacatattaaatctcattcagagtttgaagatatccaaatggtataacaacaagaaaatccaaccggtagcacaagatatgaacttagtcaagatctctggtgcgatgacaaacgcaattttcaaagttgaataccctaagttaccatcgttgctattgagaatatacggaccgaatattgataatatcattgacagggaatatgaattgcagattttggctaggctttcattgaaaaatataggtccttccctttacggctgttttgtaaacggtagatttgagcagtttctggagaattctaagactttaacaaaagacgacattagaaactggaagaactctcaaaggattgcaaggagaatgaaggagttacatgtaggtgttcctctcttgagttcagaaaggaagaacgggtcggcttgttggcaaaagattaaccagtggttgcgcacgattgagaaagtcgaccaatgggtgggggatcctaaaaacattgaaaactctttattatgtgagaattggtccaagtttatggatattgtcgatagatatcacaagtggcttatttctcaagaacagggtatagagcaagtcaacaaaaatcttatattctgccataatgatgcccaatacggcaatttacttttcactgctcctgtgatgaacacaccgagcctatacactgcaccttcgtctacatcattgacttcccaatcaagttccttatttccttcgagctccaatgtcattgtagatgatataatcaacccgccaaagcaggagcaaagccaagattccaaattggtcgtcattgattttgaatatgcaggtgccaatcccgccgcatatgatttagcgaatcatctttccgagtggatgtatgattacaacaatgctaaggccccacatcagtgccacgctgatagatatcccgataaagaacaggttttgaatttcttatactcttatgtttcgcatctaaggggtggtgctaaggaacccatagatgaagaggttcaaagactctataagtcaatcattcaatggagacccactgtacaactattttggtcgctctgggccatcctacaaagtggtaaattagagaaaaaagaagcctccactgccatcactagagaagaaattggacccaatggaaaaaaatatatcatcaagactgaacccgaatcccctgaagaagactttgttgaaaatgacgacgagcctgaagctggcgtcagcattgacacgttcgattatatggcttatggtcgtgacaagattgcggtcttttggggcgacctcattggcttaggcataatcaccgaagaagaatgcaaaaatttcagctctttcaagttcctcgatactagttatttgtaa

SEQ ID NO: 23 - DNA, 735 bp, Artificial Sequence
  Codon-optimized synthetic Thermoplasma acidophilum
atgatgattttgaaaattggtggttctgttattactgataaatctgcttatagaactgctagaacttatgctattagatctattgttaaagttttgtctggtattgaagatttggtttgtgttgttcatggtggaggttcttttggtcatattaaagctatggaatttggtttgcctggtccaaaaaacccaagatcatctatcggatattctattatccatagagatatggaaaacttggatttgatggttattgatgctatgattgaaatgggtatgagaccaatttctgttccaatttctgctttgagatatgatggtagatttgattatactccattgattagatatattgatgctggttttgttcctgtttcttatggagatgtttatattaaagatgagcattcttatggtattgtttctggtgatgatattatggctgatatggctgaattgttgaaacctgatgttgctgtttttttgactgatgttgatggtatttattctaaagatccaaaaagaaatcctgatgctgttttgttgagagatattgatactaatattacatttgatagagttcaaaatgatgttactggtggtattggaggtaaatttgaatctatggtcaaaatgaagtcttcagttaagaatggtgtttatttaattaatggtaatcatcctgaaagaattggtgatattggtaaagagtcatttatcggtactgttattaga

SEQ ID NO: 24 - DNA, 771 bp, Artificial Sequence
  Codon-optimized synthetic Methanococcoides burtonii
atgaattctaataatggtattactattttgaaaattggtggttcagttatcactgataaaagatctgaagatggtttggcttgggaagaggaaattgttagaattgctagagaaatttctggttttgaaggtaaattgattattgttcatggtgctggttcttatggtcatccacaagctaaaagatatgctttgactgaaggttttcatgctgaaggtgctgttgttactcataatgctgttaaagctttgaatagaattgttgttggtattttgaatgatgaaggtgttaatgctatttctgttcatccaatgtgttgtactgttgctaaaaatggtagaatttctgatatgtatttgggttctattagattgatgttggaaaaaggtttggttcctgttttgcatggtgatgttgttatggatgaagttaaaggtgtttctattatttctggtgatcaagttattccatatttggctactcaattgaaagcttctagaattggtgttggttctgctgctgacggtgtctttgacgataaaggtgttacaattcctgttattacttctatgaattttgatgaaattaaagcttatattggtggttcagctggtactgatgttactggtggtatgttgggtaaagttttggaaatgttggaattgggtaaaacttcttctattacttcttatatctttaatgctactgttgttggttctgtttcttcttttttgaatggtgaaaatattggtactgctattaaagattct

SEQ ID NO: 25 - DNA, 1017 bp, Artificial Sequence
  Codon-optimized synthetic Arabidopsis thaliana
atggaattgaacatctctgaaagtagaagtagaagtattagatgtatcgttaagttgggtggtgctgctattacttgtaaaaacgaattggaaaagatccacgacgaaaatttggaagttgttgcttgtcaattgagacaagcaatgttggaaggtagtgctccatctaaagtcattggtatggattggtctaaaagaccaggttctagtgaaattagttgtgatgtagatgatatcggtgatcaaaaatctagtgaattttccaaattcgtcgttgttcatggtgcaggttcttttggtcattttcaagcttcaagatctggtgttcataaaggtggtttggaaaagccaattgttaaggctggttttgttgctactagaatttctgttactaacttaaacttggaaatcgttagagctttggctagagaaggtataccaactattggtatgagtcctttttcttgtggttggtctacttctaaaagagatgttgcatccgctgatttggctactgtcgctaagacaattgattctggttttgttccagttttgcatggtgatgctgttttggataatattttgggttgtactatcttgtccggtgatgttattattagacatttggctgaccatttgaaacctgaatatgttgtttttttgaccgatgttttgggtgtttatgatagaccaccatctccttctgaacctgatgctgttttattgaaagaaattgcagttggtgaagatggtagttggaaagttgttaatccattgttggaacatactgataagaaagttgattactctgtcgctgctcatgatactactggtggtatggaaacaaaaattagtgaagctgctatgattgcaaaattgggtgttgatgtctatattgtaaaggctgctacaacacattctcaaagagctttgaatggtgatttaagagattctgtcccagaagattggttgggtactattattagattttctaagcaccatcaccatcatcactaa

SEQ ID NO: 26 - DNA, 1719 bp, Artificial Sequence
  Codon-optimized synthetic Zea mays
ggatccatgtcatacaccgttggtacctatttggccgagagattggttcaaattggtttgaaacaccactttgccgttgccggtgattataacttggttttgttggataatctgttgttgaacaaaaacatggaacaggtttattgctgtaatgagttgaattgtggtttttctgctgaaggttatgctagggctaaaggtgctgctgctgctgttgttacttattctgttggtgctttgtctgcttttgatgctattggtggtgcttatgctgagaatttgccagttattttgatttctggtgctccaaataataacgatcatgctgctggtcatgtcttgcatcatgctttgggtaaaactgattatcattatcagttggaaatggctaaaaacattacagctgctgctgaagccatttatactcccgaagaagctccagctaaaattgatcatgttattaaaaccgctctgagagaaaaaaaaccagtgtatctagaaatcgcttgcaatattgcatctatgccatgtgctgcaccaggaccagctagtgctttatttaacgacgaagcttctgacgaagcaagtttgaatgctgcagtcgaagaaactcttaaattcattgctgatagggataaagttgcagttttggttggtagtaaattgagggctgctggtgcagaggaagctgctgttaaatttgcagatgcattaggtggtgcagtagctactatggctgccgctaaaagttttttccctgaagagaatcctcattatataggtactagttggggtgaggtttcttatcctggtgttgagaaaactatgaaagaagctgatgctgttattgctttggctccagtctttaatgattatagtacaacaggttggacggatattcctgatcctaagaagttggttttggctgagccaagatcggttgtggttaatggtattaggtttccttctgttcatttgaaagattacttgactagattggctcagaaagtttctaagaaaactggtgcattggatttttttaagtcattgaatgctggtgagttgaagaaggctgcccccgctgatccttccgctccactggtcaatgctgaaatagctagacaagttgaagctctattgactccaaatactactgttatagctgaaacgggtgatagttggtttaatgctcaaagaattaagctacctaacggtgctagggtcgaatatgaaatgcaatggggtcatattggatggtctgttccagctgcttttggttatgctgttggtgctccagaaagaaggaatattttgatggttggtgatggaagttttcaattgactgctcaagaagtagctcaaatggttagattgaaaccaccagtaattattttcttgatcaacaattacggctacactattgaagttatgattcatgatggtccatataataacatcaagaattgggattacgctggattgatggaggtttttaatggtaatggcggttatgattctggagctggcaaaggcttgaaagctaaaaccggcggtgagttggctgaggcaataaaggttgctttggctaacactgatggtcccactttgattgaatgttttattggtagggaggattgtactgaagaattggtgaaatggggaaaaagagttgctgctgctaatagtagaaaaccagttaataaactgctgtaactcgag

SEQ ID NO: 27 - DNA, 2235 bp, Saccharomyces cerevisiae
atggccgctccagattatgcacttaccgatttaattgaatcggatcctcgtttcgaaagtttgaagacaagattagccggttacaccaaaggctctgatgaatatattgaagagctatactctcaattaccactgaccagctatcccaggtacaaaacatttttaaagaaacaggcggttgccatttcgaatccggataatgaagctggttttagctcgatttataggagttctctttcttctgaaaatctagtgagctgtgtggataaaaacttaagaactgcatacgatcacttcatgttttctgcaaggagatggcctcaacgtgactgtttaggttcaaggccaattgataaagccacaggcacctgggaggaaacattccgtttcgagtcgtactccacggtatctaaaagatgtcataatatcggaagtggtatattgtctttggtaaacacgaaaaggaaacgtcctttggaagccaatgattttgttgttgctatcttatcacacaacaaccctgaatggatcctaacagatttggcctgtcaggcctattctctaactaacacggctttgtacgaaacattaggtccaaacacctccgagtacatattgaatttaaccgaggcccccattctgatttttgcaaaatcaaatatgtatcatgtattgaagatggtgcctgatatgaaatttgttaatactttggtttgtatggatgaattaactcatgacgagctccgtatgctaaatgaatcgttgctacccgttaagtgcaactctctcaatgaaaaaatcacatttttttcattggagcaggtagaacaagttggttgctttaacaaaattcctgcaattccacctaccccagattccttgtatactatttcgtttacttctggtactacaggtttacctaaaggtgtggaaatgtctcacagaaacattgcgtctgggatagcatttgctttttctaccttcagaataccgccagataaaagaaaccaacagttatatgatatgtgttttttgccattggctcatatttttgaaagaatggttattgcgtatgatctagccatcgggtttggaataggcttcttacataaaccagacccaactgtattggtagaggatttgaagattttgaaaccttacgcggttgccctggttcctagaatattaacacggtttgaagccggtataaaaaacgctttggataaatcgactgtccagaggaacgtagcaaatactatattggattctaaatcggccagatttaccgcaagaggtggtccagataaatcgattatgaattttctagtttatcatcgcgtattgattgataaaatcagagactctttaggtttgtccaataactcgtttataattaccggatcagctcccatatctaaagataccttactatttttaagaagtgccttggatattggtataagacagggctacggcttaactgaaacttttgctggtgtctgtttaagcgaaccgtttgaaaaagatgtcggatcttgtggtgccataggtatttctgcagaatgtagattgaagtctgttccagaaatgggttaccatgccgacaaggatttaaaaggtgaactgcaaattcgtggcccacaggtttttgaaagatattttaaaaatccgaatgaaacttcaaaagccgttgaccaagatggttggttttccacgggagatgttgcatttatcgatggaaaaggtcgcatcagcgtcattgatcgagtcaagaactttttcaagctagcacatggtgaatatattgctccagagaaaatcgaaaatatttatttatcatcatgcccctatatcacgcaaatatttgtctttggagatcctttaaagacatttttagttggcatcgttggtgttgatgttgatgcagcgcaaccgattttagctgcaaagcacccagaggtgaaaacgtggactaaggaagtgctagtagaaaacttaaatcgtaataaaaagctaaggaaggaatttttaaacaaaattaataaatgcaccgatgggctacaaggattcgaaaaattgcataacatcaaagtcggacttgagcctttaactctcgaggatgatgttgtgacgccaacttttaaaataaagcgtgccaaagcatcaaaattcttcaaagatacattagaccaactatacgccgaaggttcactagtcaagacagaaaagctttag

SEQ ID NO: 28 - DNA, 1959 bp, Artificial Sequence
  Codon-optimized synthetic Salmonella enterica
atgtctcaaacccataagcacgctattccagctaacatcgctgatagatgtttgattaatccagaacaatatgaaaccaagtacaagcaatcaattaacgatccagatactttctggggtgaacaaggtaaaattttagactggattactccataccaaaaggttaagaacacctctttcgccccaggtaatgtctctatcaaatggtacgaagatggtactttaaacttggcagctaactgtttggatagacatttgcaagaaaacggcgatagaaccgctattatttgggaaggtgacgatacctctcaatctaagcatatctcttatagagaattgcacagagatgtttgtagatttgctaacactttgttagacttaggtattaagaagggtgacgtcgttgctatctacatgccaatggttcccgaagctgctgtcgctatgttggcctgtgcccgtattggtgccgtccattctgttatcttcggtggcttcagtccagaagctgttgctggtagaatcattgattcttcctctagattagttattactgctgatgaaggtgttagagctggtagatctattccattgaagaagaatgttgatgacgccttgaagaatccaaatgttacttccgttgaacatgttatcgttttgaaaagaactggttctgatattgattggcaagaaggtagagatctatggtggagagatttgattgaaaaagcttctcctgaacatcaaccagaagcaatgaatgctgaagatccattgtttattttgtacacttctggttccactggtaaacctaagggtgttttgcacactactggtggttacttagtttacgctgctactacctttaagtacgtttttgattatcatccaggtgacatctactggtgcactgctgatgtaggatgggttaccggtcattcctacttgctatatggcccattggcatgtggtgctactaccttgatgtttgaaggtgttccaaactggccaacccctgctagaatgtgtcaagttgttgataagcatcaagttaatattttgtacactgctccaaccgctattagagctttgatggctgaaggcgataaggctattgaaggtactgatagatcttccttgagaattttaggtagtgttggtgaaccaatcaacccagaagcctgggaatggtactggaaaaagattggtaaagaaaaatgcccagttgttgatacttggtggcaaactgaaactggtggattcatgattactcctttgcctggtgctattgaattgaaggcaggttctgctactagaccattcttcggtgtccaaccagctttggttgataacgaaggtcacccacaagaaggtgctactgaaggtaatttggttatcaccgactcttggccaggtcaagctcgtaccttgttcggcgatcatgaaagattcgaacaaacttacttttctacttttaagaacatgtacttctccggtgacggtgctagacgtgacgaagatggttactactggattacaggtcgtgttgatgatgttttgaatgtttcgggtcacagacttggtactgccgaaattgaatccgctttggtcgctcatccaaagatcgctgaagctgctgttgttggtatcccacacgctattaagggtcaagctatctatgcttacgtcaccttgaatcacggtgaagaaccatccccagaattatatgctgaagttagaaactgggttagaaaggaaatcggtcctttagctactccagacgttttgcattggacagactctttgcctaagactagatccggtaaaatcatgagaagaattttaagaaagattgctgctggtgacacttctaacttgggtgacacctctaccttggctgatccaggtgttgtcgaaaagccattagaagaaaagcaagctattgctatgccatcttaa

SEQ ID NO: 29 - DNA, 1692 bp, Saccharomyces cerevisiae
atgagattaagaaccgccattgccacactgtgcctcacggcttttacatctgcaacttcaaacaatagctacatcgccaccgaccaaacacaaaatgcctttaatgacactcacttttgtaaggtcgacaggaatgatcacgttagtcccagttgtaacgtaacattcaatgaattaaatgccataaatgaaaacattagagatgatctttcggcgttattaaaatctgatttcttcaaatactttcggctggatttatacaagcaatgttcattttgggacgccaacgatggtctgtgcttaaaccgcgcttgctctgttgatgtcgtagaggactgggatacactgcctgagtactggcagcctgagatcttgggtagtttcaataatgatacaatgaaggaagcggatgatagcgatgacgaatgtaagttcttagatcaactatgtcaaaccagtaaaaaacctgtagatatcgaagacaccatcaactactgtgatgtaaatgactttaacggtaaaaacgccgttctgattgatttaacagcaaatccggaacgatttacaggttatggtggtaagcaagctggtcaaatttggtctactatctaccaagacaactgttttacaattggcgaaactggtgaatcattggccaaagatgcattttatagacttgtatccggtttccatgcctctatcggtactcacttatcaaaggaatatttgaacacgaaaactggtaaatgggagcccaatctggatttgtttatggcaagaatcgggaactttcctgatagagtgacaaacatgtatttcaattatgctgttgtagctaaggctctctggaaaattcaaccatatttaccagaattttcattctgtgatctagtcaataaagaaatcaaaaacaaaatggataacgttatttcccagctggacacaaaaatttttaacgaagacttagtttttgccaacgacctaagtttgactttgaaggacgaattcagatctcgcttcaagaatgtcacgaagattatggattgtgtgcaatgtgatagatgtagattgtggggcaaaattcaaactaccggttacgcaactgccttgaaaattttgtttgaaatcaacgacgctgatgaattcaccaaacaacatattgttggtaagttaaccaaatatgagttgattgcactattacagactttcggtagattatctgaatctattgaatctgttaacatgttcgaaaaaatgtacgggaaaaggttaaacggttctgaaaacaggttaagctcattcttccaaaataacttcttcaacattttgaaggaggcaggcaaatcgattcgttacaccatagagaacatcaattccactaaagaaggaaagaaaaagactaacaattctcaatcacatgtatttgatgatttaaaaatgcccaaagcagaaatagttccaaggccctctaacggtacagtaaataaatggaagaaagcttggaatactgaagttaacaacgttttagaagcattcagatttatttatagaagctatttggatttacccaggaacatctgggaattatctttgatgaaggtatacaaattttggaataaattcatcggtgttgctgattacgttagtgaggagacacgagagcctatttcctataagctagatatacaataa

SEQ ID NO: 30 - DNA, 1509 bp, Saccharomyces cerevisiae
atgaaattttctgcgtatttatggtggctgtttttgaatctagcgttggtgaaaggcacttcattgctatccaacgttacattagcggaagattctttctgggagcattttcaggcttacactaatacaaagcatttaaaccaagagtggatcacaagtgaagccgtcaacaatgaaggctctaaaatatatggtgcacaatggcgactatcacagggtcgattgcaaggatccgcatgggataaaggaatcgcagttcgaacaggcaatgccgcagctatgataggacatctcttggagacacctattaatgtttcagaaacggataccttggttgtccagtacgaaattaagttggacaattctttgacgtgcggcggtgcgtttattaagttaatgtctggtttcatgaatgttgaagcattaaaacactatgcacccgatacagagggtgtcgagttagtttttggtccggattattgtgctcctgaaataaatggcgtgcaatttgccatcaataaggttgacaagatcacacatgaatctaaactaagatatttgcaagagatgcccctgtcaaaattaactgatacctcgcaatctcatctgtatacgctcataatagatgaatcagcgcagtcttttcaaattcttatagacggtaagacggttatggtaagagaacatatcgaagacaagaaaaaggtcaattttgagccacccattacaccgcctttaatgattcctgatgtttcagtagcgaaaccgcatgattgggatgatcgcatccgaatcccagatcctgaggcggtgaagctcagtgatcgggatgaacgagacccattgatgattccacatccagatggcactgaaccaccagaatggaacagctccatccccgaatacattcttgacccaaatgctcaaaagccctcgtggtggaaggaacttgagcacggggaatggataccgcccatgattaaaaatcctctttgcactgcagaacgtggttgtggccagcagataccagggctgataaataatgccaagtacaaaggtccaggcgaactcaatgaaatcataaatcccaattacatgggggaatggcatccaccggaaattgaaaacccgctatactacgaagagcagcacccattgcgcatcgaaaacgttatcagtggtgtgatcctcgagttttggagtggatctccaaacatgttgataagcaacatttatgttggtaaaaatgtaacagaggcgcaaattattgggaataagacttggctgatgagagaccgcgcgtttagaggctccgatggccccacagaacgcaaattcatgaatagcagactaggaaatctacaaacaactttccataacgaaagagaatcccctaatccatttgaccgcattatagatcgcatattagagcaacctctgaaatttgtgcttactgcggccgtcgtgctcttgacgacgtcggttctttgttgtgtagtatttacatag

SEQ ID NO: 31 - DNA, 2049 bp, Saccharomyces cerevisiae
atgtttttcaacagactaagcgctggcaagctgctggtaccactctccgtggtcctgtacgcccttttcgtggtaatattacctttacagaattctttccactcctccaatgttttagttagaggtgccgatgatgtagaaaactacggaactgttatcggtattgacttaggtactacttattcctgtgttgctgtgatgaaaaatggtaagactgaaattcttgctaatgagcaaggtaacagaatcaccccatcttacgtggcattcaccgatgatgaaagattgattggtgatgctgcaaagaaccaagttgctgccaatcctcaaaacaccatcttcgacattaagagattgatcggtttgaaatataacgacagatctgttcagaaggatatcaagcacttgccatttaatgtggttaataaagatgggaagcccgctgtagaagtaagtgtcaaaggagaaaagaaggtttttactccagaagaaatttctggtatgatcttgggtaagatgaaacaaattgccgaagattatttaggcactaaggttacccatgctgtcgttactgttcctgcttatttcaatgacgcgcaaagacaagccaccaaggatgctggtaccatcgctggtttgaacgttttgagaattgttaatgaaccaaccgcagccgccattgcctacggtttggataaatctgataaggaacatcaaattattgtttatgatttgggtggtggtactttcgatgtctctctattgtctattgaaaacggtgttttcgaagtccaagccacttctggtgatactcatttaggtggtgaagattttgactataagatcgttcgtcaattgataaaagctttcaagaagaagcatggtattgatgtgtctgacaacaacaaggccctagctaaattgaagagagaagctgaaaaggctaaacgtgccttgtccagccaaatgtccacccgtattgaaattgactccttcgttgatggtatcgacttaagtgaaaccttgaccagagctaagtttgaggaattaaacctagatctattcaagaagaccttgaagcctgtcgagaaggttttgcaagattctggtttggaaaagaaggatgttgatgatatcgttttggttggtggttctactagaattccaaaggtccaacaattgttagaatcatactttgatggtaagaaggcctccaagggtattaacccagatgaagctgttgcatacggtgcagccgttcaagctggtgtcttatccggtgaagaaggtgtcgaagatattgttttattggatgtcaacgctttgactcttggtattgaaaccactggtggtgtcatgactccattaattaagagaaatactgctattcctacaaagaaatcccaaattttctctactgccgttgacaaccaaccaaccgttatgatcaaggtatacgagggtgaaagagccatgtctaaggacaacaatctattaggtaagtttgaattaaccggcattccaccagcaccaagaggtgtacctcaaattgaagtcacatttgcacttgacgctaatggtattctgaaggtgtctgccacagataagggaactggtaaatccgaatctatcaccatcactaacgataaaggtagattaacccaagaagagattgatagaatggttgaagaggctgaaaaattcgcttctgaagacgcttctatcaaggccaaggttgaatctagaaacaaattagaaaactacgctcactctttgaaaaaccaagttaatggtgacctaggtgaaaaattggaagaagaagacaaggaaaccttattagatgctgctaacgatgttttagaatggttagatgataactttgaaaccgccattgctgaagactttgatgaaaagttcgaatctttgtccaaggtcgcttatccaattacttctaagttgtacggaggtgctgatggttctggtgccgctgattatgacgacgaagatgaagatgacgatggtgattatttcgaacacgacgaattgtag

SEQ ID NO: 32 - DNA, 1569 bp, Saccharomyces cerevisiae
atgaagttttctgctggtgccgtcctgtcatggtcctccctgctgctcgcctcctctgttttcgcccaacaagaggctgtggcccctgaagactccgctgtcgttaagttggccaccgactccttcaatgagtacattcagtcgcacgacttggtgcttgcggagttttttgctccatggtgtggccactgtaagaacatggctcctgaatacgttaaagccgccgagactttagttgagaaaaacattaccttggcccagatcgactgtactgaaaaccaggatctgtgtatggaacacaacattccagggttcccaagcttgaagattttcaaaaacagcgatgttaacaactcgatcgattacgagggacctagaactgccgaggccattgtccaattcatgatcaagcaaagccaaccggctgtcgccgttgttgctgatctaccagcttaccttgctaacgagacttttgtcactccagttatcgtccaatccggtaagattgacgccgacttcaacgccaccttttactccatggccaacaaacacttcaacgactacgactttgtctccgctgaaaacgcagacgatgatttcaagctttctatttacttgccctccgccatggacgagcctgtagtatacaacggtaagaaagccgatatcgctgacgctgatgtttttgaaaaatggttgcaagtggaagccttgccctactttggtgaaatcgacggttccgttttcgcccaatacgtcgaaagcggtttgcctttgggttacttattctacaatgacgaggaagaattggaagaatacaagcctctctttaccgagttggccaaaaagaacagaggtctaatgaactttgttagcatcgatgccagaaaattcggcagacacgccggcaacttgaacatgaaggaacaattccctctatttgccatccacgacatgactgaagacttgaagtacggtttgcctcaactctctgaagaggcgtttgacgaattgagcgacaagatcgtgttggagtctaaggctattgaatctttggttaaggacttcttgaaaggtgatgcctccccaatcgtgaagtcccaagagatcttcgagaaccaagattcctctgtcttccaattggtcggtaagaaccatgacgaaatcgtcaacgacccaaagaaggacgttcttgttttgtactatgccccatggtgtggtcactgtaagagattggccccaacttaccaagaactagctgatacctacgccaacgccacatccgacgttttgattgctaaactagaccacactgaaaacgatgtcagaggcgtcgtaattgaaggttacccaacaatcgtcttatacccaggtggtaagaagtccgaatctgttgtgtaccaaggttcaagatccttggactctttattcgacttcatcaaggaaaacggtcacttcgacgtcgacggtaaggccttgtacgaagaagcccaggaaaaagctgctgaggaagccgatgctgacgctgaattggctgacgaagaagatgccattcacgatgaattgtaa

SEQ ID NO: 33 - DNA, 3348 bp, Saccharomyces cerevisiae
atgaggttattaaggaggaatatgttagtattgacactgctcgtttgtgtgttttcatccatcatttcatgctcaatcccattgtcgtctcgcacctcaaggcggcagatagtggaagatgaagttgcctccactaaaaagctcaatttcaactatggtgtggataaaaatataaactcgcccattcctgctccaagaaccactgaaggtttaccaaatatgaaactcagctcatatccaactcctaacttattgaatactgctgataatcgacgtgctaacaaaaaaggacgtagggctgccaattctataagtgtaccctatttggagaatcgttccttgaacgaactgagtttatcagatatactaatcgcagccgacgttgagggtggacttcatgctgtagatagaagaaatggtcatatcatatggtcaatcgaaccagaaaattttcaacctctgatagaaatacaagaaccttcgaggttagaaacatatgaaacgttgattatagaacctttcggtgatgggaacatttactactttaacgcccatcaagggttacaaaaactgcctttatccatacgacaacttgtatcaacttccccgctgcacttgaaaacaaatattgtggttaatgactctggaaaaattgttgaagatgaaaaggtctacactggatcgatgagaactataatgtatactataaacatgttgaatggtgaaattatatcagcgttcggacctggttcaaaaaacgggtatttcgggagccagagtgtggattgctcacctgaggagaagataaaacttcaggaatgtgaaaatatgattgtaataggcaaaactatttttgagctgggaattcactcttatgatggagcaagctacaatgtcacttactctacatggcagcaaaatgttttagatgttcccctagcgcttcagaatacattttcaaaggacggcatgtgcatagcgcctttccgtgataaatcattgctagcaagcgatttagattttagaattgctagatgggtttctccgacattccccggaattattgttgggcttttcgatgtgtttaatgatctccgcaccaatgaaaatatactggtaccgcatccctttaatcctggtgatcatgaaagtatatcgagtaacaaagtttacttggatcagacttcgaacctctcctggtttgcattatctagtcagaattttccatctttagtcgaatcagctcccatatcaagatacgcttccagtgaccgttggagggtgtcttcaatttttgaagatgagactttattcaagaacgcaatcatgggtgttcatcagatatataataatgaatatgatcacctttatgaaaactatgaaaaaacgaatagtttggacactacgcacaaatatccacctctgatgattgattcgtccgttgatacaaccgatttacatcagaataacgagatgaattcactaaaggaatacatgtcaccagaagaccttgaggcatatagaaaaaagatacacgagcaaatatcgagagaattagatgaaaagaaccaaaattctttgctactgaagtttggaagtctagtatatcgaattatagagactggagtatttctgttgttatttctcattttttgtgcaatactacaaagattcaaaattttgccgccactatatgtattattatccaaaattggatttatgcctgaaaaggaaatccccatagttgagtcgaaatcgctaaattgtccctcttcatcggaaaatgtaaccaagccattcgatatgaaatcagggaagcaagttgtttttgaaggtgctgtgaacgatggaagtctaaaatctgaaaaagataacgatgatgctgatgaagatgatgaaaaatcactagatttaaccacagaaaagaagaagaggaaaagaggttcgagaggaggcaaaaagggccgaaaatcacgcattgcaaatataccaaactttgagcaatctttaaaaaatttggtagtatccgaaaaaattttaggttacggttcatcaggaacagtagtttttcagggaagttttcaaggaagacctgttgcggtaaagagaatgttaattgatttttgtgacatagctttaatggaaataaaacttttgactgaaagcgatgatcaccctaacgtcatacgatactactgttcagaaacaacagacagatttttgtatattgctttagagctctgcaatttgaaccttcaagatttggtggagtctaagaatgtatcagatgaaaacctgaaattacagaaagagtataatccaatttcgttattgagacaaatagcgtccggggtagcacatttacattctttaaagattatccatcgagatttaaagcctcaaaatattctcgtttctacttcgagtaggtttactgccgatcagcaaacaggagcagaaaatcttcgaattttgatatcagactttggtctttgcaaaaaactagactctggtcagtcttcatttagaacaaatttgaataacccttctggcacaagtggttggagggccccagagctgcttgaagaatcaaacaatttgcagtgccaagtcgaaacggaacactcttctagtaggcatacagtagtttcatctgattctttttatgatccgttcaccaagaggaggctaacaagatctattgatattttttctatgggatgtgtattctattatatcctatccaaagggaagcatccatttggagataaatattcacgtgaaagcaatatcataagaggaatattcagtcttgatgaaatgaaatgtctacatgatagatccttaattgcagaagctacagatctgatctcccaaatgattgatcacgatccgttaaaaagacctactgctatgaaagttctaaggcatccgttgttttggccaaagtcgaaaaaattggagttccttttaaaagttagtgataggcttgaaattgaaaacagagaccctccaagtgccctgttaatgaaatttgacgccggttctgactttgtaatacccagtggagattggactgtcaagtttgataaaacattcatggacaaccttgaaaggtacagaaaataccattcatcaaagttaatggatctattaagagcacttaggaataaatatcatcattttatggatttacctgaagatatagcagaactaatggggccggtacccgatggattttacgattacttcaccaagcgttttccaaacctattaataggtgtttatatgattgtcaaggaaaatttaagtgacgatcaaattttacgtgaatttttgtattcataa

SEQ ID NO: 34 - DNA, 717 bp, Saccharomyces cerevisiae
atggaaatgactgattttgaactaactagtaattcgcaatcgaacttggctatccctaccaacttcaagtcgactctgcctccaaggaaaagagccaagacaaaagaggaaaaggaacagcgaaggatcgagcgtattttgagaaacagaagagctgctcaccagagcagagagaaaaaaagactacatctgcagtatctcgagagaaaatgttctcttttggaaaatttactgaacagcgtcaaccttgaaaaactggctgaccacgaagacgcgttgacttgcagccacgacgcttttgttgcttctcttgacgagtacagggatttccagagcacgaggggcgcttcactggacaccagggccagttcgcactcgtcgtctgatacgttcacaccttcacctctgaactgtacaatggagcctgcgactttgtcgcccaagagtatgcgcgattccgcgtcggaccaagagacttcatgggagctgcagatgtttaagacggaaaatgtaccagagtcgacgacgctacctgccgtagacaacaacaatttgtttgatgcggtggcctcgccgttggcagacccactctgcgacgatatagcgggaaacagtctaccctttgacaattcaattgatcttgacaattggcgtaatccagaagcccagtcgggactgaactcgtttgagctcaacgacttttttataacgagttga

SEQ ID NO: 35 - DNA, 915 bp, Saccharomyces cerevisiae
atgcaacaagcaactgggaacgaattactgggtatcctagatctggataacgatatagactttgaaactgcttaccaaatgctcagcagtaacttcgacgaccaaatgtctgcgcacatacatgaaaacacgtttagtgcaacttcccctcctctgttaacacacgagctcggcataattcctaacgtagcaaccgtgcaaccctctcacgtagaaactatacctgccgataaccaaactcatcatgctcctttgcatactcatgcacactatctaaatcacaaccctcatcaaccaagcatgggttttgatcaagcgcttggtctcaagttgtctccttccagttcggggttgttgagcacgaatgaatcgaatgccattgaacagtttttagacaatctaatatcacaggatatgatgtcttccaacgcttccatgaactccgaatcacatctacatataagatcaccaaaaaagcagcataggtataccgaattaaatcaaagatatcctgaaacacatccacacagtaacacaggggagttacccacaaacacagcagatgtgccaactgagttcaccacgagggaaggacctcatcagcctatcggcaatgaccactacaacccgccaccgttttcagtacctgagatacgaatcccagactctgatattccagccaatatcgaggacgaccctgtgaaggtacggaaatggaaacacgttcaaatggagaagatacgaagaataaacaccaaagaagcctttgaaaggctcattaaatcagtaaggaccccaccgaaggaaaacgggaaaagaattcccaagcatattcttttaacttgtgtaatgaacgatatcaagtccattagaagcgcaaatgaagcactacagcacatactggatgattcctga

SEQ ID NO: 36 - DNA, 669 bp, Saccharomyces cerevisiae
atgtctagctcagaatccattagaatggtcctaattggcccacctggtgccggtaaaggtactcaagctccaaatttgcaagagcgtttccatgccgctcacttggccactggtgacatgttgagatctcaaatcgcaaagggcactcaattaggtttggaagcaaagaaaattatggaccaaggtggtttagtctctgatgacattatggttaacatgatcaaggatgaattgaccaacaatccagcttgtaagaatgggttcatcttggacggtttcccaagaaccattcctcaggctgaaaaattggaccaaatgttgaaagaacaaggaactcctttggaaaaagccatcgaattgaaggttgatgatgaattgttggttgccagaattaccggtagattaattcacccagcctctggcagatcctaccacaagatctttaacccaccaaaggaagacatgaaggatgacgtcaccggtgaagctttagttcaaagatctgatgacaatgcagacgccttgaagaagagattagctgcttaccatgctcaaaccgaaccaattgttgacttttacaaaaagaccggtatctgggctggtgttgatgcttcccaacctcctgctactgtttgggctgacatcttgaacaagctaggtaaggattaa

SEQ ID NO: 37 - DNA, 1011 bp, Artificial Sequence
  Codon-optimized synthetic Pseudomonas stutzeri
atgttgccaaagttggttattactcatagagttcatgatgaaattttgcaattgttggctccacattgtgaattgatgactaaccaaactgattctactttgactagagaagaaatcttgagaagatgtagagatgctcaagctatgatggctttcatgccagatagagttgatgctgatttcttgcaagcttgtccagaattgagagttgttggttgtgctttgaagggttttgataatttcgatgttgacgcttgtaccgctagaggtgtttggttaactttcgttccagatttgttgacagttccaactgctgaattggcaattggtttggctgttggtttgggtagacatttgagagctgctgatgccttcgttagatctggtgaatttcaaggttggcaacctcaattttatggtactggtttagataacgctactgttggtatcttgggtatgggtgctattggtttggctatggccgatagattacaaggttggggtgctacattgcaataccatgaagctaaggctttagatactcaaactgaacaaagattgggtttgagacaagttgcttgttcagaattattcgcttcttctgatttcatcttgttggctttgccattgaacgctgatactcaacatttggttaacgctgaattgctagctttagttagaccaggtgctttgttggttaatccatgtagaggttctgttgttgacgaagctgcagttttagctgctttggaaagaggtcaattgggtggttacgctgccgacgtttttgaaatggaagattgggctagagctgatagacctagattgattgatccagctttgttggctcatccaaatacattgtttactccacacattggttccgctgttagagctgttagattagaaattgaaagatgtgctgctcaaaatatcattcaagttttggctggtgccagaccaattaacgctgctaatagattgccaaaggctgaaccagctgcttgttaa

SEQ ID NO: 38 - DNA, 441 bp, Artificial Sequence
  Codon-optimized synthetic Vitreoscilla stercoraria
atgttggatcaacaaactattaacattattaaggctactgttccagttttgaaggaacatggtgttactattactactactttttacaagaacttgttcgctaagcatccagaagttagaccattgtttgatatgggtagacaagaatctttggaacaaccaaaggctttagctatgactgttttggctgctgctcaaaacattgaaaacttgccagctattttgccagctgttaagaagattgctgttaagcattgtcaagctggtgttgctgctgctcattacccaattgttggtcaagaattgttgggtgctattaaggaagttttgggtgacgctgctactgatgatattttggatgcttggggtaaagcttacggtgttattgctgatgttttcattcaagttgaagctgatttgtacgctcaagctgttgaataa

SEQ ID NO: 39 - DNA, 1257 bp, Saccharomyces cerevisiae
atgtcaggaacattcaatgatataagaagaaggaagaaggaagaaggaagccctacagccggtattaccgaaaggcatgagaataagtctttgtcaagcatcgataaaagagaacagactctcaaaccacaactagagtcatgctgtccattggcgaccccttttgaaagaaggttacaaactctggctgtagcatggcacacttcttcatttgtactcttctccatatttacgttatttgcaatctcgacaccagcactgtgggttcttgctattccatatatgatttatttttttttcgataggtctcctgcaactggcgaagtggtaaatcgatactctcttcgatttcgttcattgcccatttggaagtggtattgtgattatttccctataagtttgattaaaactgtcaatttaaaaccaacttttacgctttcaaaaaataagagagttaacgaaaaaaattacaagattagattgtggccaactaagtattccattaatctcaaaagcaactctactattgactatcgcaaccaggaatgtacagggccaacgtacttatttggttaccatccacacggcataggagcacttggtgcgtttggagcgtttgcaacagaaggttgtaactattccaagattttcccaggtattcctatttctctgatgacactggtcacacaatttcatatcccattgtatagagactacttattggcgttaggtatttcttcagtatctcggaaaaacgctttaaggactctaagcaaaaatcagtcgatctgcattgttgttggtggcgctagggaatctttattaagttcaacaaatggtacacaactgattttaaacaaaagaaagggttttattaaactggccattcaaacggggaatattaacctagtgcctgtgtttgcatttggagaggtggactgttataatgttctgagcacaaaaaaagattcagtcctgggtaaaatgcaactatggttcaaagaaaactttggttttaccattcccattttctacgcaagaggattattcaattacgatttcggtttgttgccatttagagcgcctatcaatgttgttgttggaaggcctatatacgttgaaaagaaaataacaaatccgccagatgatgttgttaatcatttccatgatttgtatattgcggagttgaaaagactatattacgaaaatagagaaaaatatggggtaccggatgcagaattgaagatagttgggtaa

SEQ ID NO: 40 - DNA, 2529 bp, Saccharomyces cerevisiae
gctacgctatcaggtgctatagatgtcattgtagtggagcatccagacggaaggctatcatgttctccctttcatgtgaggttcggcaaatttcaaattctaaagccatctcaaaagaaagtccaagtgtttataaatgagaaactgagtaatatgccaatgaaactgagtgattctggagaagcctatttcgttttcgagatgggtgaccaggtcactgatgtccctgacgaattgcttgtgtcgcccgtgatgagcgccacatcaagcccccctcaatcacctgaaacatccatcttagaaggaggaaccgagggtgaaggtgaaggtgaaaatgaaaataagaagaaggaaaagaaagtgctagaggaaccagattttttagatatcaatgacactggagattcaggcagtaaaaatagtgaaactacagggtcgctttctcctactgaatcctctacaacgacaccaccagattcagttgaagagaggaagcttgttgagcagcgtacaaagaactttcagcaaaaactaaacaaaaaactcactgaaatccatatacccagtaaacttgataacaatggcgacttactactagacactgaaggttacaagccaaacaagaatatgatgcatgacacagacatacaactgaagcagttgttaaaggacgaattcggtaatgattcagatatttccagttttatcaaggaggacaaaaatggcaacatcaagatcgtaaatccttacgagcaccttactgatttatctcctccaggtacgcctccaacaatggccacaagcggatcagttttaggcttagatgcaatggaatcaggaagtactttgaattcgttatcttcttcaccttctggttccgatactgaggacgaaacatcatttagcaaagaacaaagcagtaaaagtgaaaaaactagcaagaaaggaacagcagggagcggtgagaccgagaaaagatacatacgaacgataagattgactaatgaccagttaaagtgcctaaatttaacttatggtgaaaatgatctgaaattttccgtagatcacggaaaagctattgttacgtcaaaattattcgtttggaggtgggatgttccaattgttatcagtgatattgatggcaccatcacaaaatcggacgctttaggccatgttctggcaatgataggaaaagactggacgcacttgggtgtagccaagttatttagcgagatctccaggaatggctataatatactctatctaactgcaagaagtgctggacaagctgattccacgaggagttatttgcgatcaattgaacagaatggcagcaaactaccaaatgggcctgtgattttatcacccgatagaacgatggctgcgttaaggcgggaagtaatactaaaaaaacctgaagtctttaaaatcgcgtgtctaaacgacataagatccttgtattttgaagacagtgataacgaagtggatacagaggaaaaatcaacaccattttttgccggctttggtaataggattactgatgctttatcttacagaactgtggggatacctagttcaagaattttcacaataaatacagagggtgaggttcatatggaattattggagttagcaggttacagaagctcctatattcatatcaatgagcttgtcgatcatttctttccaccagtcagccttgatagtgtcgatctaagaactaatacttccatggttcctggctccccccctaatagaacgttggataactttgactcagaaattacttcaggtcgcaaaacgctatttagaggcaatcaggaagagaaattcacagacgtaaatttttggagagacccgttagtcgacatcgacaacttatcggatattagcaatgatgattctgataacatcgatgaagatactgacgtatcacaacaaagcaacattagtagaaatagggcaaattcagtcaaaaccgccaaggtcactaaagccccgcaaagaaatgtgagcggcagcacaaataacaacgaagttttagccgcttcgtctgatgtagaaaatgcgtctgacctggtgagttcccatagtagctcaggatccacgcccaataaatctacaatgtccaaaggggacattggaaaacaaatatatttggagctaggttctccacttgcatcgccaaaactaagatatttagacgatatggatgatgaagactccaattacaatagaactaaatcaaggagagcatcttctgcagccgcgactagtatcgataaagagttcaaaaagctctctgtgtcaaaggccggcgctccaacaagaattgtttcaaagatcaacgtttcaaatgacgtacattcacttgggaattcagataccgaatcacgaagggagcaaagtgttaatgaaacagggcgcaatcagctaccccacaactcaatggacgataaagatttggattcaagagtaagcgatgaattcgatgacgatgaattcgacgaagatgaattcgaagattaa

SEQ ID NO: 41 - DNA, 1176 bp, Saccharomyces cerevisiae
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

(19) United States
(12) Patent Application Publication
(10) Pub. No.: US 2023/0167473 A1
(43) Pub. Date: Jun. 1, 2023

(54) METHOD FOR PRODUCING HETEROGENOUS CANNABICHROMENE FROM SACCHAROMYCES CEREVISIAE

(71) Applicant: DALIAN UNIVERSITY OF TECHNOLOGY, Dalian (CN)

(72) Inventors: Chuang XUE, Dalian (CN); Mingming QI, Dalian (CN); Wenqiang ZHANG, Dalian (CN)

(21) Appl. No.: 17/780,423

(22) PCT Filed: Nov. 17, 2021
(86) PCT No.: PCT/CN2021/131047

§ 371 Date: May 26, 2022

(30) Foreign Application Priority Data
Dec. 14, 2020 (CN) 202011475558.7

(51) Int. Cl. C12P 17/06; C12N 15/52; C12N 9/02; C12N 9/04; C12N 9/10; C12N 9/88; C12N 9/00

(52) U.S. Cl. CPC C12P 17/06; C12N 15/52; C12N 9/0004; C12Y 121/99; C12N 9/0006; C12Y 101/01157; C12N 9/001; C12Y 103/01044; C12N 9/1029; C12Y 203/01009; C12N 9/88; C12Y 402/01017; C12Y 404/01026; C12N 9/93; C12Y 604/01002

(57) ABSTRACT

A recombinant host cell capable of biosynthesizing cannabichromenic acid and a construction method thereof, and a method for biosynthesizing cannabichromenic acid through the recombinant host cell. Saccharomyces cerevisiae is taken as a host. First, cannabigerolic acid synthase and cannabichromenic acid synthase are over-expressed in the host; then, a metabolic pathway of a precursor compound, olivetolic acid, synthesizing cannabichromenic acid from saccharides is constructed in the host, a metabolic pathway for hexanoic acid to olivetolic acid is further constructed in the host, an endogenous mevalonate pathway of the host and a metabolic pathway of acetyl-CoA are optimized, cannabichromenic acid synthase is rationally designed, highly active cannabichromenic acid synthase is screened out, and finally, a cannabichromene pathway is located to peroxisomes and lipid droplets by using the cell compartmentalization principle to obtain recombinant Saccharomyces cerevisiae capable of biosynthesizing cannabichromenic acid.

The instant application contains a Sequence Listing which has been submitted electronically in the ASCII text file and is hereby incorporated by reference in its entirety. The ASCII text file is a sequence listing entitled “2022-07-22 Sequence Listing” created on Jul. 22, 2022 and having a size of 104 KBs in compliance of 37 CFR 1.821.

### TECHNICAL FIELD

The present invention belongs to the field of biotechnology and medicine, and particularly relates to a host cell capable of biosynthesizing cannabichromene (CBC) and a construction method thereof, and a biosynthesis method of cannabichromene.

### BACKGROUND

Cannabis sativa has been used for thousands of years because of containing a variety of pharmacologically active cannabinoids. More than 113 of cannabinoids have been isolated and identified from the cannabis plant and divided into different types such as cannabigerols (CBGs), cannabichromenes (CBCs), cannabidiols (CBDs), Δ⁹-tetrahydrocannabinols (Δ⁹-THCs), Δ8-tetrahydrocannabinols (Δ⁸-THCs), cannabicyclols (CBLs), cannabielsoins (CBEs), cannabinols (CBNs), cannabinodiols (CBNDs), cannabitriols (CBTs) and other cannabinoids (Elsohly, M. A.; Slade, D., Chemical constituents of marijuana: the complex mixture of natural cannabinoids. Life Sciences 2005, 78 (5), 539-48.), wherein the cannabichromene (CBC) and the acid form thereof of cannabichromenic acid as well as cannabigerol (CBG) and the acid form thereof of cannabigerolic acid are main components of cannabinoid, and these acid cannabinoids are decarboxylated in the conditions of heating or long term storage to form neural cannabinoids (for example, cannabichromene is formed by cannabichromenic acid, and cannabigerolic acid is formed by cannabigerol). Researches show that cannabichromene (CBC) and cannabigerol (CBG) have antibacterial activity on Staphylococcus aureus (Appendino, G.; Gibbons, S.; Giana, A.; Pagani, A.; Grassi, G.; Stavri, M.; Smith, E.; Rahman, M. M., Antibacterial cannabinoids from Cannabis sativa: a structure-activity study. Journal of Natural Products 2008, 71 (8), 1427-30.). CBG also has significant activity on several ligand-gated cation channels of the TRP superfamily, and can serve as an agonist of TRPV1 (TRP vanillin 1) and TRPA1 (TRP ankyrin 1) as well as an active inhibitor of TRPM8 (TRP melatonin 8) (Pollastro, F.; Taglialatela-Scafati, O.; Allarà, M.; Muñoz, E.; Di Marzo, V.; De Petrocellis, L.; Appendino, G., Bioactive prenylogous cannabinoid from fiber hemp (Cannabis sativa). Journal of Natural Products 2011, 74 (9), 2019-22.). CBC can inhibit inactivation of endocannabinoids and activate TRPA1, thereby producing protective effect against intestinal inflammation in experimental model systems. In addition, CBC has multiple pharmacologic and biological effects, including pain relieving, anti-nociception, and anti-inflammatory activity.

At present, cannabichromene (CBC) and cannabigerol (CBG) are obtained mainly by extraction from plants or chemical synthesis. However, the chemical synthesis process is complex, the cost is high, and the yield is very low. The agricultural planting of Cannabis saliva faces several challenges, for example, susceptibility of plants to climate and diseases, no GAP standardization, low content of cannabichromene and cannabigerol in Cannabis sativa, large occupied area of cultivated land, long cycle, coexistence with other abundant cannabinoids, time and labor consuming in obtaining pure samples from plants, and strong impact on researches on treatment potential.

Microbial fermentation has the advantages of high production efficiency and short cycle, a method for producing a large number of high value-added products from cheap carbon sources is provided, and meanwhile, it is the research hotspot in recent years to obtain microorganisms with specific functions through transformation of the metabolic pathway of microorganisms based on the genetic engineering technology. At present, the related researches on host cells capable of biosynthesizing cannabichromenic acid obtained by means of genetic engineering, a construction method thereof and applications thereof on biosynthesis of cannabichromenic acid have been not reported.

In another aspect, in order to further increase the flux of GPP, a synthesis pathway for biosynthesizing isoprenoid from secondary substrate is called isopentenyl alcohol use pathway (IUP), which is used for producing isoprenoid precursors IPP and DMAPP from isoprenol or prenol. IUP is a metabolic pathway, the flux thereof can be competitive with some fastest isoprenoid pathways, and separation of isoprenoid biosynthesis and central carbon metabolism is allowed, which will greatly simplify the engineering effort in future production of high value isoprenoid. Isopentenyl alcohol isomers are selected as precursors of IPP and DMAPP due to similar structure. First, isoprenol or isopentenyl alcohol is respectively phosphorylated to form isopentenyl phosphate (IP) or dimethylallyl phosphate (DMAP); and then, IP or DMAP is phosphorylated again to form IPP or DMAPP. The second step of the pathway is catalysis with isopentenyl phosphate kinase (IPK) which is a part of the archaebacteria mevalonate pathway. Although the primary phosphorylation does not occur in nature, some phosphokinases can exhibit confounding kinase activity. Therefore, several kinases, including IPK homologue, are screened in accordance with the isopentenyl alcohol kinase activity in the present invention, and some IPK variants can convert isopentenyl alcohol into DMAP through the confounding activity.

However, the competition pathways and metabolic crosstalk often prevent effective synthesis of target compounds in the cytoplasm. Eukaryotic cells control the metabolic complexity thereof by isolating biochemical pathways through organelles. In Saccharomyces cerevisiae, peroxisomes are appropriate targets of organelle engineering, which are not necessary for cell viability in most of culture conditions, and the number and size thereof can be modified in various ways to better meet the needs of engineering. Yeast peroxisomes are the site of β-oxidation of fatty acid, forming an acetyl-CoA library, which can provide isoprenoid precursors IPP and DMAPP through heterologous pathways such as mevalonate (MVA) pathway. In addition, peroxisome has a simple layer film, which allows a great number of small molecule compounds to penetrate passively or through channel protein. Meanwhile, peroxisome is also a detoxifying organelle, which can treat and isolate more toxic molecules of the remaining part of a cell. Moreover, cannabinoids, as hydrophobic compounds, have limited solubility in cytoplasm, but have high solubility in hydrophobic liquids such as lipid, oil or fat. The precursor substance CBGA synthesizing cannabichromenic acid is inferred to be possibly isolated in lipid droplets. Therefore, cannabichromenic acid synthase polypeptide is located to the lipid droplets in order to obtain higher reaction rate and higher productivity by increasing the local concentration of substrate and enzyme.

### SUMMARY

To solve the problems of complexity, high cost and low yield of the preparation method of cannabichromene and cannabichromenic acid, the present invention provides the following technical solution.

The present invention constructs a recombinant Saccharomyces cerevisiae strain capable of biosynthesizing cannabichromenic acid. The biosynthetic pathway of cannabichromenic acid comprises a plurality of polypeptides. In one aspect, the precursor, geranyl pyrophosphate (GPP), synthesizing cannabigerolic acid from Saccharomyces cerevisiae by monosaccharides through the endogenous mevalonate pathway is optimized; in another aspect, a metabolic pathway for hexanoic acid to hexanoyl-CoA is constructed, hexanoic acid is converted to hexanoyl-CoA through acyl activating enzyme polypeptide (CsAAE1) by replenishing hexanoic acid, acetyl-CoA is catalyzed by acetyl-CoA carboxylase polypeptide (ACC1) to produce malonyl-CoA, and olivetolic acid (OA) is produced from hexanoyl-CoA and trimolecular malonyl-CoA through polyketide synthase polypeptide (CsTKS) and olivetolic acid cyclase polypeptide (CsOAC); and moreover, metabolic pathways of β-ketothiolase polypeptide (RebktB), 3-hydroxybutyryl coenzyme A dehydrogenase polypeptide (CnpaaH1), crotonase polypeptide (Cacrt) and trans-2-enoyl-CoA reductase polypeptide (Tdter) are also constructed so that hexanoyl-CoA is produced from Saccharomyces cerevisiae by monosaccharides through acetyl-CoA, and then olivetolic acid (OA) is produced through polyketide synthase polypeptide (CsTKS) and olivetolic acid cyclase polypeptide (CsOAC), thereby increasing the yield of olivetolic acid. Finally, cannabigerolic acid (CBGA) is produced from geranyl pyrophosphate (GPP) and olivetolic acid (OA) through cannabigerolic acid synthase polypeptide (CsPT4), and cannabichromenic acid (CBCA) is produced through catalysis of cannabichromenic acid synthase (as shown in FIG. 1).

The present invention provides a recombinant Saccharomyces cerevisiae strain synthesizing cannabichromenic acid (CBCA), by which cannabigerolic acid synthase polypeptide (CsPT4) and cannabichromenic acid synthase polypeptide (CBCAS) are heterogeneously expressed.

Further, β-ketothiolase polypeptide (RebktB), 3-hydroxybutyryl coenzyme A dehydrogenase polypeptide (CnpaaH1), crotonase polypeptide (Cacrt), trans-2-enoyl-CoA reductase polypeptide (Tdter), type III polyketide synthase polypeptide (CsTKS) and olivetolic acid cyclase polypeptide (CsOAC) are over-expressed by the recombinant Saccharomyces cerevisiae strain.

Further, acyl activating enzyme polypeptide (CsAAE1) and acetyl-CoA carboxylase polypeptide (ACC1) are over-expressed by the recombinant Saccharomyces cerevisiae strain.

Further, truncated HMG-CoA reductase polypeptide (tHMG1), acetoacetyl-CoA thiolase polypeptide (mvaE), HMG-CoA synthase polypeptide (mvaS), mutants of farnesyl pyrophosphate synthetase polypeptide (ERG20mut), mevalonate kinase polypeptide (ERG12), phosphomevalonate kinase polypeptide (ERG8), mevalonate pyrophosphate decarboxylase polypeptide (ERG19) and isopentenyl-pyrophosphate delta isomerase polypeptide (IDI) are over-expressed by the recombinant Saccharomyces cerevisiae strain.

Further, acetaldehyde dehydrogenase polypeptide (ALD6), acetyl-CoA synthetase polypeptide (ACS2) and alcohol dehydrogenase polypeptide (ADH2) are over-expressed by the recombinant Saccharomyces cerevisiae strain.

Further, cannabigerolic acid synthase polypeptide, cannabichromenic acid synthase polypeptide, β-ketothiolase polypeptide, 3-hydroxybutyryl coenzyme A dehydrogenase polypeptide, crotonase polypeptide, trans-2-enoyl-CoA reductase polypeptide, type III polyketide synthase polypeptide, olivetolic acid cyclase polypeptide, acyl activating enzyme polypeptide, acetyl-CoA carboxylase polypeptide, truncated HMG-CoA reductase polypeptide, acetoacetyl-CoA thiolase polypeptide, HMG-CoA synthase polypeptide, mutants of farnesyl pyrophosphate synthetase polypeptide, mevalonate kinase polypeptide (ERG12), phosphomevalonate kinase polypeptide (ERG8), mevalonate pyrophosphate decarboxylase polypeptide, isopentenyl-pyrophosphate delta isomerase polypeptide, acetaldehyde dehydrogenase polypeptide, acetyl-CoA synthetase polypeptide and alcohol dehydrogenase polypeptide are homologous or heterogenous.

Further, the cannabigerolic acid synthase polypeptide is derived from Cannabis sativa, and the cannabichromenic acid synthase polypeptide is derived from Cannabis sativa; the β-ketothiolase polypeptide is derived from Ralstonia eutropha, the 3-hydroxybutyryl coenzyme A dehydrogenase polypeptide is derived from Cupriavidus necator, the crotonase polypeptide is derived from Clostridium acetobutylicum, and the trans-2-enoyl-CoA reductase polypeptide is derived from Treponema denticola; the type III polyketide synthase polypeptide is derived from Cannabis sativa, and the olivetolic acid cyclase polypeptide is derived from Cannabis sativa; the acyl activating enzyme polypeptide is derived from Cannabis sativa, the acetyl-CoA carboxylase polypeptide is derived from Saccharomyces cerevisiae, the truncated truncated HMG-CoA reductase polypeptide is derived from Saccharomyces cerevisiae, the acetoacetyl-CoA thiolase polypeptide is derived from Enterococcus faecalis, the HMG-CoA synthase polypeptide is derived from Enterococcus faecalis, the mutants of farnesyl pyrophosphate synthetase polypeptide is derived from Saccharomyces cerevisiae, the mevalonate kinase polypeptide (ERG12) and the phosphomevalonate kinase polypeptide (ERG8) are derived from Saccharomyces cerevisiae, the mevalonate pyrophosphate decarboxylase polypeptide is derived from Saccharomyces cerevisiae, and the isopentenyl-pyrophosphate delta isomerase polypeptide is derived from Saccharomyces cerevisiae; and the acetaldehyde dehydrogenase polypeptide, the acetyl-CoA synthetase polypeptide and the alcohol dehydrogenase polypeptide are derived from Saccharomyces cerevisiae.

Further, the nucleotide sequence of the cannabigerolic acid synthase polypeptide is shown as SEQ ID NO:1, and the nucleotide sequence of the cannabichromenic acid synthase polypeptide is shown as SEQ ID NO:2; the nucleotide sequence of the β-ketothiolase polypeptide is shown as SEQ ID NO:3, the nucleotide sequence of the 3-hydroxybutyryl coenzyme A dehydrogenase polypeptide is shown as SEQ ID NO:4, the nucleotide sequence of the crotonase polypeptide is shown as SEQ ID NO:5, the nucleotide sequence of the trans-2-enoyl-CoA reductase polypeptide is shown as SEQ ID NO:6, the nucleotide sequence of the type III polyketide synthase polypeptide is shown as SEQ ID NO:7, and the nucleotide sequence of the olivetolic acid cyclase polypeptide is shown as SEQ ID NO:8; the nucleotide sequence of the acyl activating enzyme polypeptide is shown as SEQ ID NO:9, and the nucleotide sequence of the acetyl-CoA carboxylase polypeptide is shown as SEQ ID NO:10; the nucleotide sequence of the truncated HMG-CoA reductase polypeptide is shown as SEQ ID NO:11, the nucleotide sequence of the acetoacetyl-CoA thiolase polypeptide is shown as SEQ ID NO:12, the nucleotide sequence of the HMG-CoA synthase polypeptide is shown as SEQ ID NO:13, the nucleotide sequence of the mutants of farnesyl pyrophosphate synthetase polypeptide is shown as SEQ ID NO:14, the nucleotide sequence of the mevalonate kinase polypeptide (ERG12) is shown as SEQ ID NO:15, the nucleotide sequence of the phosphomevalonate kinase polypeptide (ERG8) is shown as SEQ ID NO:16, the nucleotide sequence of the mevalonate pyrophosphate decarboxylase polypeptide is shown as SEQ ID NO:17, and the nucleotide sequence of the isopentenyl-pyrophosphate delta isomerase polypeptide is shown as SEQ ID NO:18; and the nucleotide sequence of the acetaldehyde dehydrogenase polypeptide is shown as SEQ ID NO:19, the nucleotide sequence of the acetyl-CoA synthetase polypeptide is shown as SEQ ID NO:20, and the nucleotide sequence of the alcohol dehydrogenase polypeptide is shown as SEQ ID NO:21.

Further, the cannabigerolic acid synthase polypeptide, the cannabichromenic acid synthase polypeptide, the β-ketothiolase polypeptide, the 3-hydroxybutyryl coenzyme A dehydrogenase polypeptide, the crotonase polypeptide, the trans-2-enoyl-CoA reductase polypeptide, the type III polyketide synthase polypeptide, the olivetolic acid cyclase polypeptide, the acyl activating enzyme polypeptide, the acetyl-CoA carboxylase polypeptide, the truncated HMG-CoA reductase polypeptide, the acetoacetyl-CoA thiolase polypeptide, the HMG-CoA synthase polypeptide, the mutants of farnesyl pyrophosphate synthetase polypeptide, the mevalonate kinase polypeptide (ERG12), the phosphomevalonate kinase polypeptide (ERG8), the mevalonate pyrophosphate decarboxylase polypeptide, the isopentenyl-pyrophosphate delta isomerase polypeptide, the acetaldehyde dehydrogenase polypeptide, the acetyl-CoA synthetase polypeptide and the alcohol dehydrogenase polypeptide respectively have at least 70%, 80%, 90% or 95% homology with nucleotides shown as SEQ ID NO:1-21 and have the activity function of the corresponding enzymes.

Further, the cannabigerolic acid synthase polypeptide, the cannabichromenic acid synthase polypeptide, the β-ketothiolase polypeptide, the 3-hydroxybutyryl coenzyme A dehydrogenase polypeptide, the crotonase polypeptide, the trans-2-enoyl-CoA reductase polypeptide, the type III polyketide synthase polypeptide, the olivetolic acid cyclase polypeptide, the acyl activating enzyme polypeptide, the acetyl-CoA carboxylase polypeptide, the truncated HMG-CoA reductase polypeptide, the acetoacetyl-CoA thiolase polypeptide, the HMG-CoA synthase polypeptide, the mutants of farnesyl pyrophosphate synthetase polypeptide, the mevalonate kinase polypeptide (ERG12), the phosphomevalonate kinase polypeptide (ERG8), the mevalonate pyrophosphate decarboxylase polypeptide, the isopentenyl-pyrophosphate delta isomerase polypeptide, the acetaldehyde dehydrogenase polypeptide, the acetyl-CoA synthetase polypeptide and the alcohol dehydrogenase polypeptide respectively have nucleotide sequences obtained by nucleotides shown as SEQ ID NO:1-21 through replacement, substitution or deletion of one or more nucleotide sequences and have the activity function of the corresponding enzymes.

Further, the cannabigerolic acid synthase polypeptide, the cannabichromenic acid synthase polypeptide, the β-ketothiolase polypeptide, the 3-hydroxybutyryl coenzyme A dehydrogenase polypeptide, the crotonase polypeptide, the trans-2-enoyl-CoA reductase polypeptide, the type III polyketide synthase polypeptide, the olivetolic acid cyclase polypeptide, the acyl activating enzyme polypeptide, the acetyl-CoA carboxylase polypeptide, the truncated HMG-CoA reductase polypeptide, the acetoacetyl-CoA thiolase polypeptide, the HMG-CoA synthase polypeptide, the mutants of farnesyl pyrophosphate synthetase polypeptide, the mevalonate kinase polypeptide (ERG12), the phosphomevalonate kinase polypeptide (ERG8), the mevalonate pyrophosphate decarboxylase polypeptide, the isopentenyl-pyrophosphate delta isomerase polypeptide, the acetaldehyde dehydrogenase polypeptide, the acetyl-CoA synthetase polypeptide and the alcohol dehydrogenase polypeptide respectively have nucleotide sequences hybridized and complemented with the nucleotide sequences shown as SEQ ID NO:1-21 in moderate or high or extremely high conditions and have the activity function of the corresponding enzymes.

Further, the nucleotide sequences of the cannabigerolic acid synthase polypeptide, the cannabichromenic acid synthase polypeptide, the β-ketothiolase polypeptide, the 3-hydroxybutyryl coenzyme A dehydrogenase polypeptide, the crotonase polypeptide, the trans-2-enoyl-CoA reductase polypeptide, the type III polyketide synthase polypeptide, the olivetolic acid cyclase polypeptide, the acyl activating enzyme polypeptide, the acetyl-CoA carboxylase polypeptide, the truncated HMG-CoA reductase polypeptide, the acetoacetyl-CoA thiolase polypeptide, the HMG-CoA synthase polypeptide, the mutants of farnesyl pyrophosphate synthetase polypeptide, the mevalonate kinase polypeptide (ERG12), the phosphomevalonate kinase polypeptide (ERG8), the mevalonate pyrophosphate decarboxylase polypeptide, the isopentenyl-pyrophosphate delta isomerase polypeptide, the acetaldehyde dehydrogenase polypeptide, the acetyl-CoA synthetase polypeptide and the alcohol dehydrogenase polypeptide are nucleotide sequences codon-optimized in part or in whole.

Further, the cannabigerolic acid synthase polypeptide insertion locus is located at the 416d locus, the CAN1y locus and the YOLCd1b locus of yeast genome; and the cannabichromenic acid synthase polypeptide insertion locus is located at the 308a locus, the HIS3b locus and the 511b locus of the yeast genome. The β-ketothiolase polypeptide insertion locus is located at the SAP155b locus of the yeast genome; the insertion loci of the 3-hydroxybutyryl coenzyme A dehydrogenase polypeptide and the crotonase polypeptide are located at the SAP155c locus of the yeast genome; the trans-2-enoyl-CoA reductase polypeptide insertion locus is located at the YPRCδ15c locus of the yeast genome; the insertion loci of the type III polyketide synthase polypeptide and the olivetolic acid cyclase polypeptide are located at the 1622b locus, the X4 locus, the XI locus 3 and the X115 locus of the yeast genome; the acyl activating enzyme polypeptide insertion locus is located at the 911b locus of the yeast genome; the acetyl-CoA carboxylase polypeptide insertion locus is located at the X3 locus of the yeast genome; the insertion loci of the truncated HMG-CoA reductase polypeptide and the mutant geranyl diphosphate synthase ERG20mut (F96W, N127W) are located at the 1021b locus of the yeast genome; the insertion loci of the acetoacetyl-CoA thiolase polypeptide and the HMG-CoA synthase polypeptide are located at the 1414a locus of the yeast genome; the insertion loci of the mevalonate kinase polypeptide (ERG12) and the isopentenyl-pyrophosphate delta isomerase polypeptide are located at the 1114a locus of the yeast genome; the insertion loci of the phosphomevalonate kinase polypeptide (ERG8) and the mevalonate pyrophosphate decarboxylase polypeptide are located at the 1014a locus of the yeast genome; the insertion loci of the acetaldehyde dehydrogenase polypeptide and the acetyl-CoA synthetase polypeptide are located at the 1309a locus of the yeast genome; and the alcohol dehydrogenase polypeptide insertion locus is located at the X2 locus of the yeast genome.

Further, the copy number of the above polypeptides is respectively 1-10.

The present invention in another aspect provides a construction method of the above recombinant Saccharomyces cerevisiae strain, mainly comprising the following steps:

1) Respectively constructing expression cassettes of cannabigerolic acid synthase polypeptide and cannabichromenic acid synthase polypeptide, and inserting the above expression cassettes into a Saccharomyces cerevisiae genome through the homologous recombination technology;

2) Respectively constructing expression cassettes of β-ketothiolase polypeptide, 3-hydroxybutyryl coenzyme A dehydrogenase polypeptide, crotonase polypeptide, trans-2-enoyl-CoA reductase polypeptide, type III polyketide synthase polypeptide and olivetolic acid cyclase polypeptide, and inserting the above expression cassettes into the Saccharomyces cerevisiae genome obtained in step (1) through homologous recombination;

3) Respectively constructing expression cassettes of acyl activating enzyme polypeptide and acetyl-CoA carboxylase polypeptide, and inserting the above expression cassettes into the Saccharomyces cerevisiae genome obtained in step (2) through homologous recombination;

4) Respectively constructing expression cassettes of truncated HMG-CoA reductase polypeptide, acetoacetyl-CoA thiolase polypeptide, HMG-CoA synthase polypeptide, mutants of farnesyl pyrophosphate synthetase polypeptide, mevalonate kinase polypeptide (ERG12), phosphomevalonate kinase polypeptide (ERG8), mevalonate pyrophosphate decarboxylase polypeptide and isopentenyl-pyrophosphate delta isomerase polypeptide, and inserting the above expression cassettes into the Saccharomyces cerevisiae genome obtained in step (3) through homologous recombination;

5) Respectively constructing expression cassettes of acetaldehyde dehydrogenase polypeptide, acetyl-CoA synthetase polypeptide and alcohol dehydrogenase polypeptide, and inserting the above expression cassettes into the Saccharomyces cerevisiae genome obtained in step (4) through homologous recombination.

Further, zinc finger nuclease (ZFN), transcription activator-like effector nuclease (TALEN) and the CRISPR/Cas system are used for homologous recombination.

Further, a CRISPR/Cas system is used for the homologous recombination.

Further, promoters of the above polypeptides are respectively constitutive promoters or inducible promoters.

Further, the promoter is GAL1, GAL10, GPD, TEF1, PGK1 or ADH.

Further, bioinformatics analysis and sequence promoter deletion analysis show that YPL062W is the core promoter of ALD6, and the expression level of ALD6 is negatively correlated with the yield of terpenoids. In order to further increase the flux of acetyl-CoA, the alcohol dehydrogenase polypeptide (ADH1) and YPL062W are knocked out; and pyruvate decarboxylase polypeptide (PDC) from Zea mays, endogenous acyl-CoA synthetase polypeptide (FAA2), and acetyl-CoA synthetase polypeptide (SeACS) from Salmonella enterica are over-expressed.

In order to increase the flux of GPP and each downstream product in the recombinant Saccharomyces cerevisiae strain of cannabichromenic acid, the present invention provides another construction method of the above recombinant Saccharomyces cerevisiae strain, mainly comprising the following steps:

(1) In one aspect, screening out promiscuous kinase polypeptide to be used from Salmonella enterica subsp (PhoN) and Saccharomyces cerevisiae (ScCK). In another aspect, screening out isopentenyl phosphate kinase polypeptide which can be successfully expressed in the Saccharomyces cerevisiae from multiple species of Thermoplasma acidophilum (thaIPK), Methanococcus vannielii (mvIPK), Methanolobus tindarius (mt IPK), Methanosalsum zhilinae (mzIPK), Methanococcus maripaludis (mmIPK), Methanococcoides burtonii (mbIPK) and Arabidopsis thaliana (atIPK).

(2) Locating the promiscuous kinase polypeptide screened out, isopentenyl phosphate kinase polypeptide, acetoacetyl-CoA thiolase polypeptide, HMG-CoA synthase polypeptide, mevalonate kinase polypeptide (ERG12), phosphomevalonate kinase polypeptide (ERG8), mevalonate pyrophosphate decarboxylase polypeptide, isopentenyl-pyrophosphate delta isomerase polypeptide (IDI), mutants of farnesyl pyrophosphate synthetase polypeptide (ERG20mut), cannabigerolic acid synthase polypeptide (CsPT4) and cannabichromenic acid synthase polypeptide (CBCAS) to peroxisomes.

(3) In order to increase the number and volume of the peroxisomes, knocking out peroxisomal membrane protein PEX31 and PEX32; and over-expressing peroxisomal membrane protein PEX3, PEX19, PEXI1 and PEX34 so as to increase the production of cannabichromenic acid.

Further, in order to ensure the proper folding of CBCAS, a variety of molecular chaperones, foldases and transcription activators are screened simultaneously to improve the expression activity of CBCAS, including proteins (CNE1p, KAR2p, PDIIp and EROIp) involved in folding and endoplasmic reticulum quality control, cofactor (FADIp), UPR protein IRE1*p, UPR activator Hac1s and endoplasmic reticulum size regulator INO1.

Further, a plurality of mutants, respectively H292L, H292R, Y417H, Y417R, N89Q-N499Q, R463C-D488C, T379S, K377R, N196Q, F171Y, S170T, R349K, F365Y, V138A, R532K, L524I, Y472F, N528Q, F353Y and a combination of double mutants, triple mutants and multiple mutants, are produced by site-directed mutagenesis of cannabichromenic acid synthase, and the mutant with the best activity is screened out to produce cannabichromenic acid.

Further, in order to increase intracellular ATP and dissolved oxygen supply, the adenylate kinase polypeptide (ADK1), phosphite dehydrogenase polypeptide ptxD from Pseudomonas stutzeri (ADK1) and Vibrio hyaluronicus hemoglobin polypeptide (VHB) are over-expressed to increase the synthesis rate of ATP, so as to promote cell growth and cannabinoid production.

Further, cannabichromenic acid synthase polypeptide is located to the lipid droplets in order to obtain higher reaction rate and higher productivity by increasing the local concentration of substrate and enzyme. Meanwhile, in order to improve the solubility of cannabinoid, the key enzyme polypeptides of DAG acyltransferase polypeptide DGA1, G3P dehydrogenase polypeptide GPD1 and phosphatidic acid phosphatase polypeptide (PAH1) in the triacylglycerol pathway TAG are over-expressed, and the key protein polypeptide SEI1 for lipid droplet synthesis is knocked out to increase lipid level and lipid droplet aggregation. Meanwhile, cannabichromenic acid synthase polypeptide is located to the lipid droplets in order to obtain higher reaction rate and higher productivity and expand the storage capacity of engineered yeast to synthesize cannabinoids by increasing the local concentration of substrate and enzyme.

Further, the key enzyme polypeptides in the metabolic pathway of cannabichromene are simultaneously integrated to the rDNA locus of Saccharomyces cerevisiae to achieve multi-copy expression of a plurality of key enzyme polypeptides.

Further, in order to save fermentation cost, Gal80 is knocked out to remove the inhibitory effect thereof on Gal4, and meanwhile, the promoter of Gal4 is replaced to remove the inhibitory effect of glucose. Therefore, galactose is no longer needed as an inducer, and the yield of cannabichromenic acid (CBCA) is optimized through mixed fermentation of glucose and ethanol.

Further, promiscuous kinase polypeptide, isopentenyl phosphate kinase polypeptide, pyruvate decarboxylase polypeptide, acyl-CoA synthetase polypeptide, related polypeptides involved in folding and endoplasmic reticulum quality control, adenylate kinase polypeptide, phosphite dehydrogenase polypeptide, phosphatidic acid phosphatase polypeptide, DAG acyltransferase polypeptide, G3P dehydrogenase polypeptide, phosphatidic acid phosphatase polypeptide, endoplasmic reticulum quality control proteins (CNE1p, KAR2p, PDI1p and ERO1p), cofactor (FAD1p), UPR protein IRE1*p, UPR activator Hac1s and endoplasmic reticulum size regulator INO1 are homologous or heterogenous.

Further, the promiscuous kinase polypeptide is derived from Saccharomyces cerevisiae; the isopentenyl phosphate kinase polypeptide is derived from Thermoplasma acidophilum, Methanococcoides methylutens and Arabidopsis thaliana; the pyruvate decarboxylase polypeptide is derived from corn; the acyl-CoA synthetase polypeptide is derived from Saccharomyces cerevisiae; the acetyl-CoA synthetase polypeptide is derived from Salmonella; the related polypeptides involved in folding and endoplasmic reticulum quality control and the adenylate kinase polypeptide are derived from Saccharomyces cerevisiae; the phosphite dehydrogenase polypeptide is derived from Pseudomonas stutzeri; and the phosphatidic acid phosphatase polypeptide is derived from Vibrio hyaluronicus, and the DAG acyltransferase polypeptide, the G3P dehydrogenase polypeptide, the phosphatidic acid phosphatase polypeptide, the endoplasmic reticulum quality control proteins (CNE1p, KAR2p, PDI1p and ERO1p), the cofactor (FAD1p), the UPR protein IRE1*p, the UPR activator Hac1s and the endoplasmic reticulum size regulator INO1 are derived from Saccharomyces cerevisiae.

Further, the nucleotide sequence of the promiscuous kinase polypeptide (ScCK) is shown as SEQ ID NO:22, the nucleotide sequence of the isopentenyl phosphate kinase polypeptide (thaIPK) is shown as SEQ ID NO:23, the nucleotide sequence of the isopentenyl phosphate kinase polypeptide (mbIPK) is shown as SEQ ID NO:24, the nucleotide sequence of the isopentenyl phosphate kinase polypeptide (atIPK) is shown as SEQ ID NO:25, the nucleotide sequence of the pyruvate decarboxylase polypeptide (ZmPDC) is shown as SEQ ID NO:26, the nucleotide sequence of the acyl-CoA synthetase polypeptide is shown as SEQ ID NO:27, the nucleotide sequence of the acetyl-CoA synthetase polypeptide (SeACS) is shown as SEQ ID NO:28, the nucleotide sequence of the polypeptide (ERO1) involved in folding and endoplasmic reticulum quality control is shown as SEQ ID NO:29, the nucleotide sequence of the polypeptide (CEN1) involved in folding and endoplasmic reticulum quality control is shown as SEQ ID NO:30, the nucleotide sequence of the polypeptide (KAR2) involved in folding and endoplasmic reticulum quality control is shown as SEQ ID NO:31, the nucleotide sequence of the polypeptide (PDI1) involved in folding and endoplasmic reticulum quality control is shown as SEQ ID NO:32, the nucleotide sequence of unfolded protein response (UPR) polypeptide (IRE1*) is shown as SEQ ID NO:33, the nucleotide sequence of unfolded protein response (UPR) polypeptide (HAC1s) is shown as SEQ ID NO:34, the nucleotide sequence of the endoplasmic reticulum size related polypeptide (INO1) is shown as SEQ ID NO:35, the nucleotide sequence of the adenylate kinase polypeptide is shown as SEQ ID NO:36, the nucleotide sequence of the phosphite dehydrogenase polypeptide ptxD is shown as SEQ ID NO:37, the nucleotide sequence of the phosphatidic acid phosphatase polypeptide is shown as SEQ ID NO:38, the nucleotide sequence of the DAG acyltransferase polypeptide is shown as SEQ ID NO:39, the nucleotide sequence of the phosphatidic acid phosphatase polypeptide is shown as SEQ ID NO:40, and the nucleotide sequence of the G3P dehydrogenase polypeptide is shown as SEQ ID NO:41.

Further, the nucleotide sequences of the promiscuous kinase polypeptide, the isopentenyl phosphate kinase polypeptide, the pyruvate decarboxylase polypeptide, the acyl-CoA synthetase polypeptide, the related polypeptides involved in folding and endoplasmic reticulum quality control, the adenylate kinase polypeptide, the phosphite dehydrogenase polypeptide, the phosphatidic acid phosphatase polypeptide, the DAG acyltransferase polypeptide, the G3P dehydrogenase polypeptide, the phosphatidic acid phosphatase polypeptide, the endoplasmic reticulum quality control proteins (CNE1p, KAR2p, PDI1p and ERO1p), the cofactor (FAD1p), the UPR protein IRE1*p, the UPR activator Hac1s and the endoplasmic reticulum size regulator INO1 respectively have at least 70%, 80%, 90% or 95% homology with nucleotides shown as SEQ ID NO:22-41 and have the activity function of the corresponding enzymes.

Further, the nucleotide sequences of the promiscuous kinase polypeptide, the isopentenyl phosphate kinase polypeptide, the pyruvate decarboxylase polypeptide, the acyl-CoA synthetase polypeptide, the related polypeptides involved in folding and endoplasmic reticulum quality control, the adenylate kinase polypeptide, the phosphite dehydrogenase polypeptide, the phosphatidic acid phosphatase polypeptide, the DAG acyltransferase polypeptide, the G3P dehydrogenase polypeptide, the phosphatidic acid phosphatase polypeptide, the endoplasmic reticulum quality control proteins (CNE1p, KAR2p, PDI1p and ERO1p), the cofactor (FAD1p), the UPR protein IRE1*p, the UPR activator Hac1s and the endoplasmic reticulum size regulator INO1 are respectively nucleotide sequences obtained by nucleotides shown as SEQ ID NO:22-41 through replacement, substitution or deletion of one or more nucleotide sequences and have the activity function of the corresponding enzymes.

Further, the nucleotide sequences of the promiscuous kinase polypeptide, the isopentenyl phosphate kinase polypeptide, the pyruvate decarboxylase polypeptide, the acyl-CoA synthetase polypeptide, the related polypeptides involved in folding and endoplasmic reticulum quality control, the adenylate kinase polypeptide, the phosphite dehydrogenase polypeptide, the phosphatidic acid phosphatase polypeptide, the DAG acyltransferase polypeptide, the G3P dehydrogenase polypeptide, the phosphatidic acid phosphatase polypeptide, the endoplasmic reticulum quality control proteins (CNE1p, KAR2p, PDI1p and ERO1p), the cofactor (FAD1p), the UPR protein IRE1*p, the UPR activator Hac1s and the endoplasmic reticulum size regulator INO1 are respectively nucleotide sequences hybridized and complemented with the nucleotide sequences shown as SEQ ID NO:22-41 in moderate or high or extremely high conditions and have the activity function of the corresponding enzymes.

Further, the nucleotide sequences of the promiscuous kinase polypeptide, the isopentenyl phosphate kinase polypeptide, the pyruvate decarboxylase polypeptide, the acyl-CoA synthetase polypeptide, the related polypeptides involved in folding and endoplasmic reticulum quality control, the adenylate kinase polypeptide, the phosphite dehydrogenase polypeptide, the phosphatidic acid phosphatase polypeptide, the DAG acyltransferase polypeptide, the G3P dehydrogenase polypeptide, the phosphatidic acid phosphatase polypeptide, the endoplasmic reticulum quality control proteins (CNE1p, KAR2p, PDI1p and ERO1p), the cofactor (FAD1p), the UPR protein IRE1*p, the UPR activator Hac1s and the endoplasmic reticulum size regulator INO1 are nucleotide sequences codon-optimized in part or in whole.

Further, the EfmvaE-EfmvaS-SKL insertion locus is located at the YIRCA6 locus of the Saccharomyces cerevisiae genome; the ERG19-ERG8-SKL insertion locus is located at the YMRWΔ15 locus of the Saccharomyces cerevisiae genome; the ERG12-SKL insertion locus is located at the YNRCΔ9 locus of the Saccharomyces cerevisiae genome; the ScCK-SKL insertion locus is located at the YGLCτ3 locus of the Saccharomyces cerevisiae genome; the atIPK-SKL insertion locus is located at the YORWΔ17 locus of the Saccharomyces cerevisiae genome; the IDI-SKL insertion locus is located at the YPRCτ3 locus of the Saccharomyces cerevisiae genome; the ERG20 mut (F96W, N127W)-SKL insertion locus is located at the SPB1/PBN1 locus of the Saccharomyces cerevisiae genome; the zmPDC insertion locus is located at the YCRWδ11 locus of the Saccharomyces cerevisiae genome; the FAA2 insertion locus is located at the XII4 locus of the Saccharomyces cerevisiae genome; the acetyl-CoA synthetase polypeptide (SeACS) insertion locus is located at the YORWΔ22 locus of the Saccharomyces cerevisiae genome; the ADK1 insertion locus is located at the YARCδ8 locus of the Saccharomyces cerevisiae genome; the ptxD insertion locus is located at the YCRWδ11 locus of the Saccharomyces cerevisiae genome; the VHB insertion locus is located at the YBRWδ16 locus of the Saccharomyces cerevisiae genome; the CNE1 insertion locus is located at the I12 locus of the Saccharomyces cerevisiae genome; the KAR2 insertion locus is located at the I4 and I32 loci of the Saccharomyces cerevisiae genome; the PDI1 insertion locus is located at the I10 locus of the Saccharomyces cerevisiae genome; the ERO1 insertion locus is located at the X3 locus of the Saccharomyces cerevisiae genome; the IRE1 insertion locus is located at the I8 locus of the Saccharomyces cerevisiae genome; the Hac1s insertion locus is located at the I28 locus of the Saccharomyces cerevisiae genome; the INO1 insertion locus is located at the I3 locus of the Saccharomyces cerevisiae genome; the DGA1 insertion locus is located at the YCRWδ12 locus of the Saccharomyces cerevisiae genome; the PAH1 insertion locus is located at the YERCδ8 locus of the Saccharomyces cerevisiae genome; and the GPD1 insertion locus is located at the YORWΔ22 locus of the Saccharomyces cerevisiae genome.

Further, the above key enzyme polypeptides are constructed into high-copy plasmid vectors for free expression.

The present invention provides a method for producing cannabichromenic acid by fermenting the above recombinant Saccharomyces cerevisiae, which mainly comprises the following steps:

1) Culturing cells of the above recombinant Saccharomyces cerevisiae in an appropriate culture medium for a period of time;

2) Recycling cannabichromenic acid generated by fermentation;

3) Decarboxylating cannabichromenic acid by heating or long term storage to form cannabichromene.

Further, the culture medium is a YPD culture medium.

Further, the culture medium contains one or a mixture of more of glucose, galactose, glycerine, ethanol, starch, hexanoic acid and olivetolic acid.

Further, the culture conditions are as follows: the revolving speed is 50-300 rpm, the temperature is 28-32° C., and the culture time is 24-120 h.

Further, the process of recycling cannabichromenic acid generated by fermentation comprises the step of extracting cannabichromenic acid from fermentation liquor with an organic solvent.

Further, the organic solvent is one or a mixture of more of ethyl acetate, hexane, heptane, petroleum ether and chloroform.

Further, the process of recycling cannabichromenic acid generated by fermentation comprises the step of crushing recombinant Saccharomyces cerevisiae obtained by fermentation.

Further, the crushing method is high-pressure homogenization crushing, ultrasonic crushing, ball-milling brushing, repeated freezing and thawing crushing or enzymatic solubilization crushing.

Further, the volume ratio of the organic solvent to the fermentation liquor in the extraction process is 1:1-1:20.

Compared with the prior art, the present invention has the following beneficial effects:

1. The present invention discloses a recombinant Saccharomyces cerevisiae strain capable of biosynthesizing cannabichromenic acid, and provides a new approach for producing a large amount of high value-added cannabichromene from cheap carbon sources.

2. The method for constructing the recombinant Saccharomyces cerevisiae strain capable of biosynthesizing cannabichromenic acid of the present invention is accurate and efficient, and the recombinant Saccharomyces cerevisiae strain obtained has stable genetic performance.

3. The method for producing cannabichromenic acid by fermenting recombinant Saccharomyces cerevisiae disclosed by the present invention has high production efficiency, short cycle and low cost and is beneficial to mass production of cannabichromene and extension of application in the field of medicine.

### DESCRIPTION OF DRAWINGS

FIG. 1 shows a biosynthetic pathway of cannabichromenic acid by recombinant Saccharomyces cerevisiae, wherein CsAAE1: acyl activating enzyme polypeptide, ACC1: acetyl-CoA carboxylase polypeptide, RebktB: β-ketothiolase polypeptide, CnpaaH1: 3-hydroxybutyryl coenzyme A dehydrogenase polypeptide, Cacrt: crotonase polypeptide, Tdter: trans-2-enoyl-CoA reductase polypeptide, CsTKS: polyketide synthase polypeptide, CsOAC: olivetolic acid cyclase polypeptide, CsPT4: olivetolic acid geranyl transferase, and CBCAS: cannabichromenic acid synthase polypeptide.

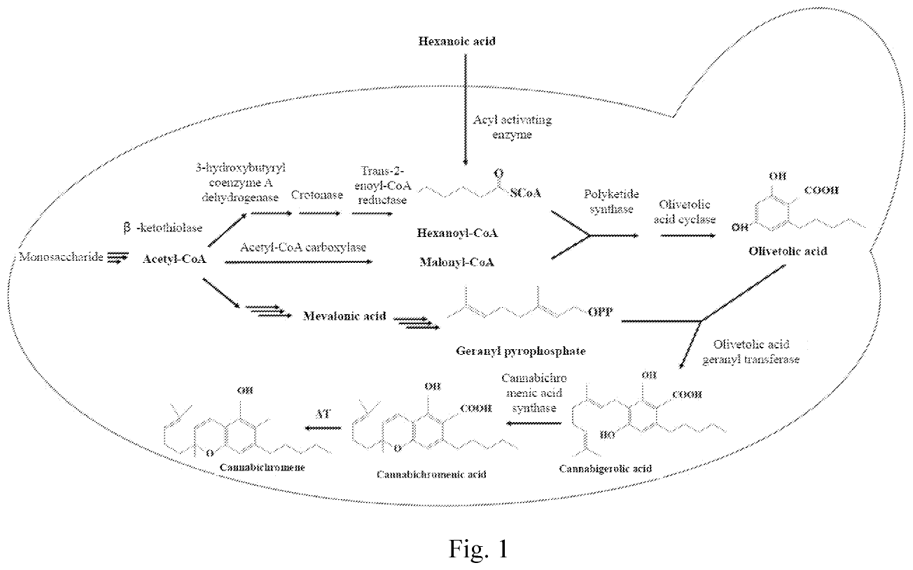

FIG. 2 is a schematic diagram of a GPP biosynthetic pathway.

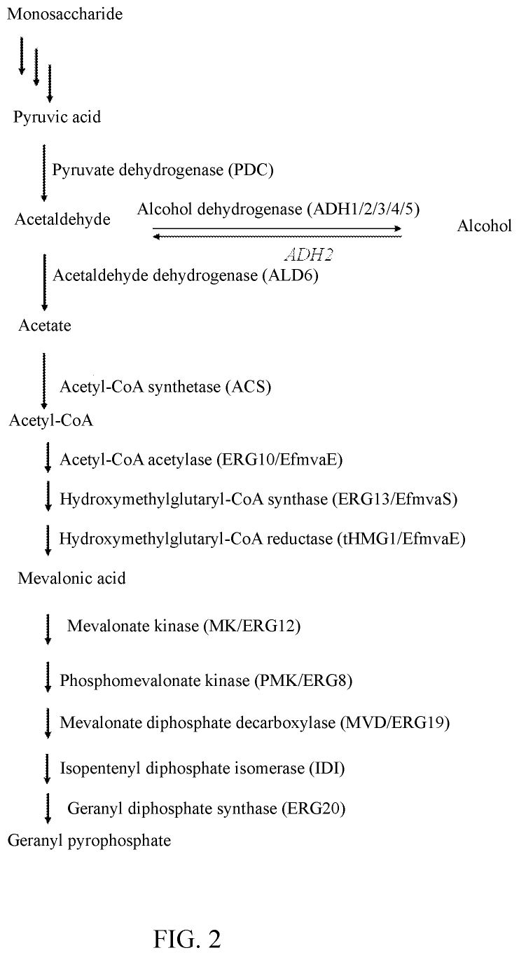

FIG. 3 is a liquid chromatogram of cannabichromenic acid produced by fermenting recombinant Saccharomyces cerevisiae, wherein the upper figure is an LC-MS liquid chromatogram of cannabichromenic acid standard, the abscissa is retention time, and the ordinate is abundance; and the lower figure is an LC-MS liquid chromatogram of a fermentation sample of a recombinant genetically engineered strain of cannabichromenic acid.

FIG. 4 is a mass spectrum of cannabichromenic acid produced by fermenting recombinant Saccharomyces cerevisiae, wherein the abscissa is M/Z, and the ordinate is abundance. The upper figure is an LC-MS mass spectrogram of cannabichromenic acid standard; and the lower figure is an LC-MS mass spectrogram of a fermentation sample of a recombinant genetically engineered strain of cannabichromenic acid.

### DETAILED DESCRIPTION

The present invention is described below in detail in combination with embodiments, but the embodiments of the present invention are limited thereto. Obviously, the embodiments in the following description are merely part of the embodiments of the present invention, and for those skilled in the art, other similar embodiments obtained without contributing creative labor fall into the protection scope of the present invention.

### Embodiment 1

Construction of Recombinant Yeast Strain Capable of Expressing Cannabigerolic Acid Synthase Polypeptide and Cannabichromenic Acid Synthase Polypeptide

The host bacterium of the present invention is Saccharomyces cerevisiae INVSc1, diplontic INVSc1 has high robustness, and the complicated gene regulatory network thereof is beneficial to expression and catalysis of enzymes in adverse environmental conditions. The present invention constructs two gene expression cassettes: a codon-optimized CsPT4 gene expression cassette and a codon-optimized CBCAS gene expression cassette, based on the findings that cannabigerolic acid (CBGA) is produced by geranyl pyrophosphate (GPP) and olivetolic acid (OA) under the action of cannabigerolic acid synthase polypeptide (CsPT4) and cannabichromenic acid (CBCA) is formed by cannabigerolic acid (CBGA) under the catalysis of cannabichromenic acid synthase polypeptide (CBCAS), a GAL10 promoter and a CYC1 terminator are used for the CsPT4 gene expression cassette, a GAL10 promoter and a CYC1 terminator are used for the CBCAS gene expression cassette, and the CsPT4 gene expression cassette is integrated to 416d, CAN1y and YOLCd1b genome loci of Saccharomyces cerevisiae through the genome editing technology; and the CBCAS gene expression cassette is respectively integrated to 308a, HIS3b and 511b genome loci of Saccharomyces cerevisiae to express three copies of CsPT4 and CBCAS, so as to obtain the recombinant Saccharomyces cerevisiae strain capable of expressing cannabigerolic acid synthase polypeptide and cannabichromenic acid synthase polypeptide.

### Embodiment 2

Construction of Recombinant Yeast Strain Capable of Producing Cannabichromenic Acid from Saccharides

In order to enable the biosynthesis of olivetolic acid in the recombinant Saccharomyces cerevisiae in embodiment 1, a biometabolic synthesis pathway by which olivetolic acid is produced from saccharides through hexanoyl-CoA is constructed in the above recombinant Saccharomyces cerevisiae. Six codon-optimized gene expression cassettes: a RebktB gene expression cassette, a CnpaaH1 gene expression cassette, a Cacrt gene expression cassette, a Tdter gene expression cassette, a CsTKS gene expression cassette and a CsOAC gene expression cassette, are constructed. A TEF1 promoter and a TEF1 terminator are used for the RebktB gene expression cassette, a GAL10 promoter and a CYC1 terminator are used for the CnpaaH1 gene expression cassette, a GAL1 promoter and an ADH1 terminator are used for the Cacrt gene expression cassette, a PGK1 promoter and an HXT7 terminator are used for the Tdter gene expression cassette, a GAL10 promoter and a CYC1 terminator are used for the CsTKS gene expression cassette, and a GAL1 promoter and an ADH1 terminator are used for the CsOAC gene expression cassette. Through the gene editing technology, the RebktB gene expression cassette is integrated to the SAP155b genome locus of Saccharomyces cerevisiae, the CnpaaH1 gene expression cassette and the Cacrt gene expression cassette are integrated to the SAP155c genome locus of Saccharomyces cerevisiae, and the Tdter gene expression cassette is integrated to the YPRCδ15c genome locus of Saccharomyces cerevisiae, respectively expressing one copy of the above polypeptides. The CsOAC gene expression cassette and the CsTKS gene expression cassette are formed into one expression cassette to be respectively integrated to 1622b, X4, XI3 and XII5 genome loci of Saccharomyces cerevisiae to express four copies of the above polypeptides, finally obtaining the recombinant Saccharomyces cerevisiae strain capable of producing cannabichromenic acid from saccharides.

### Embodiment 3

Construction of Biometabolic Synthesis Pathway for Hexanoic Acid to Olivetolic Acid in Recombinant Saccharomyces cerevisiae

In order to increase the flux of hexanoyl-CoA of the recombinant Saccharomyces cerevisiae in embodiment 2, a biometabolic synthesis pathway for hexanoic acid to olivetolic acid in the recombinant Saccharomyces cerevisiae is constructed, two gene expression cassettes: a codon-optimized CsAAE1 gene expression cassette and a codon-optimized ACC1 gene expression cassette, are constructed, a GPD promoter and a CYC1 terminator are used for the CsAAE1 and ACC1 gene expression cassettes, and the ACC1 gene expression cassette is integrated to the X3 genome locus of Saccharomyces cerevisiae through the gene editing technology to over-express one copy of the above polypeptides; and the CsAAE1 gene expression cassette is integrated to 208a, 911b and 106a genome loci of Saccharomyces cerevisiae to express three copies of the above polypeptides, and hexanoic acid is converted to hexanoyl-CoA by feeding hexanoic acid under the catalysis of CsAAE1. Meanwhile, acetyl-CoA is catalyzed by ACC1 to produce malonyl-CoA, and olivetolic acid is produced by hexanoyl-CoA and malonyl-CoA under the action of type III polyketide synthase polypeptide (CsTKS) and olivetolic acid cyclase polypeptide (CsOAC) so as to increase the yield of cannabichromenic acid biosynthesized by recombinant Saccharomyces cerevisiae.

### Embodiment 4

Optimization of Endogenous Mevalonate Pathway of Recombinant Saccharomyces cerevisiae

In order to increase the flux of GPP of the recombinant Saccharomyces cerevisiae in embodiment 3, the endogenous mevalonate pathway of the recombinant Saccharomyces cerevisiae is further optimized, and eight gene expression cassettes: a truncated tHMG1 gene expression cassette, a codon-optimized mvaE gene expression cassette, an mvaS gene expression cassette, an ERG20mut (F96W, N127W) gene expression cassette, an ERG12 gene expression cassette, an ERG8 gene expression cassette, an ERG19 gene expression cassette and an IDI gene expression cassette, are constructed. A GAL1 promoter and an ADH1 terminator are used for the tHMG1 gene expression cassette, a GAL1 promoter and an ADH1 terminator are used for the mvaE gene expression cassette, a GAL10 promoter and a CYC1 terminator are used for the mvaS gene expression cassette, a GAL10 promoter and a CYC1 terminator are used for the ERG20mut gene expression cassette, a GAL10 promoter and a CYC1 terminator are used for the ERG12 gene expression cassette, a GAL1 promoter and an ADH1 terminator are used for the ERG8 gene expression cassette, a GAL10 promoter and a CYC1 terminator are used for the ERG19 gene expression cassette, and a GAL1 promoter and an ADH1 terminator are used for the IDI gene expression cassette. Through the gene editing technology, the tHMG1 gene expression cassette and the ERG20mut gene expression cassette are integrated to the 1021b genome locus of Saccharomyces cerevisiae, the mvaE gene expression cassette and the mvaS gene expression cassette are integrated to the 1414a genome locus of Saccharomyces cerevisiae, the ERG12 gene expression cassette and the IDI gene expression cassette are integrated to the 1114a genome locus of Saccharomyces cerevisiae, and the ERG8 gene expression cassette and the ERG19 gene expression cassette are integrated to the 1014a genome locus of Saccharomyces cerevisiae to over-express one copy of the above polypeptides, so as to ensure the supply of geranyl pyrophosphate in the mevalonate downstream pathway in the recombinant Saccharomyces cerevisiae.

### Embodiment 5

Optimization of Metabolic Pathway of Acetyl-CoA of Recombinant Saccharomyces cerevisiae

In order to increase the metabolic flux of acetyl-CoA in the cytoplasm of the recombinant Saccharomyces cerevisiae in embodiment 4, acetaldehyde dehydrogenase polypeptide (ALD6) from Saccharomyces cerevisiae, acetyl-CoA synthetase polypeptide (ACS2) and alcohol dehydrogenase polypeptide (ADH2) are obtained from the NCBI database, and three gene expression cassettes: an ALD6 gene expression cassette, an ACS2 gene expression cassette and an ADH2 gene expression cassette, are constructed. A GAL10 promoter and a CYC1 terminator are used for the ALD6 gene expression cassette, a GAL1 promoter and an ADH1 terminator are used for the ACS2 gene expression cassette, and a GPD promoter and a CYC1 terminator are used for the ADH2 gene expression cassette. Through the gene editing technology, the ALD6 gene expression cassette and the ACS2 gene expression cassette are integrated to the 1309a genome locus of Saccharomyces cerevisiae, and the ADH2 gene expression cassette is integrated to the X2 genome locus of Saccharomyces cerevisiae to over-express one copy of the above polypeptides, so as to increase the flux of acetyl-CoA in the cytoplasm of the recombinant Saccharomyces cerevisiae and provide precursor compounds for biosynthesis of mevalonate pathway, olivetolic acid and cannabichromenic acid.

### Embodiment 6

Construction of Biosynthetic Pathway of Cannabichromene in Peroxisome

First, a gene expression cassette which can be located to peroxisome is constructed; and for codon-optimized IPK gene, choline kinase (ScCK), acetoacetyl-CoA thiolase polypeptide (EfmvaE), HMG-CoA synthase polypeptide (EfmvaS), mevalonate kinase polypeptide (ERG12), phosphomevalonate kinase polypeptide (ERG8), mevalonate pyrophosphate decarboxylase polypeptide (ERG19), isopentenyl-pyrophosphate delta isomerase polypeptide (IDI), mutants of farnesyl pyrophosphate synthetase polypeptide (ERG20mut), codon-optimized cannabigerolic acid synthase polypeptide (CsPT4) and cannabichromenic acid synthase polypeptide (CBCAS), a PTS1 signal peptide sequence KL-X5-QL of peroxisome is added to N terminals, and a PTS2 signal peptide sequence SKL of peroxisome is added to C terminals. A TEF1 promoter and a TEF1 terminator are used for the IPK and choline kinase (ScCK) gene expression cassettes. A GAL10 promoter and a CYC1 terminator are both used for the EfmvaE, EfmvaS, ERG8, ERG19, ERG12, IDI, ERG20mut, CsPT4 and CBCAS gene expression cassettes. Through the gene editing technology, the EfmvaE-EfmvaS-SKL gene expression cassette is integrated to the YIRCA6 genome locus of Saccharomyces cerevisiae; the ERG19-ERG8-SKL gene expression cassette is integrated to the YMRWΔ15 genome locus of Saccharomyces cerevisiae; the ERG12-SKL gene expression cassette is integrated to the YNRCΔ9 genome locus of Saccharomyces cerevisiae; the ScCK-SKL gene expression cassette is integrated to the YGLCτ3 genome locus of Saccharomyces cerevisiae; the atIPK-SKL gene expression cassette is integrated to the YORWΔ17 genome locus of Saccharomyces cerevisiae; the IDI-SKL gene expression cassette is integrated to the YPRCτ3 genome locus of Saccharomyces cerevisiae; and the ERG20 mut (F96W, N127W)-SKL gene expression cassette is integrated to the SPB1/PBN1 genome locus of Saccharomyces cerevisiae.

### Embodiment 7

In order to further the supply of acetyl-CoA in cytoplasm, the pyruvate decarboxylase polypeptide (PDC) from Zea mays and the endogenous acyl-CoA synthetase polypeptide (FAA2) are over-expressed. The codon-optimized pyruvate decarboxylase polypeptide zmPDC, the endogenous acyl-CoA synthetase polypeptide (FAA2) and the acetyl-CoA synthetase polypeptide (SeACS) from Salmonella enterica are respectively constructed to a TEF1 promoter and a TEF1 terminator. Through the gene editing technology, the zmPDC, FAA2 and acetyl-CoA synthetase polypeptide (SeACS) expression cassettes are respectively integrated to X2, XII4 and YORWΔ22 genome loci of Saccharomyces cerevisiae, and meanwhile, ADH1 is knocked out.

### Embodiment 8

In order to increase intracellular ATP and dissolved oxygen supply, adenylate kinase polypeptide (ADK1), phosphite dehydrogenase polypeptide ptxD from Pseudomonas stutzeri and Vibrio hyaluronicus hemoglobin polypeptide (VHB) are over-expressed. The adenylate kinase polypeptide (ADK1), the codon-optimized phosphite dehydrogenase polypeptide ptxD and the Vibrio hyaluronicus hemoglobin polypeptide (VHB) are respectively constructed to a TEF1 promoter and a TEF1 terminator. Through the gene editing technology, the ADK1, ptxD and VHB gene expression cassettes are respectively integrated to YARC88, YCRWSI1 and YBRW816 genome loci of Saccharomyces cerevisiae.

### Embodiment 9

In order to ensure the proper folding of CBCAS, proteins (CNE1p, KAR2p, PDI1p, ERO1p and IRE1p) involved in folding and endoplasmic reticulum quality control, cofactor (FAD1p), UPR activator Hac1s and endoplasmic reticulum size regulator INO1 are amplified from the Saccharomyces cerevisiae genome. Expression cassettes are constructed respectively by using PGK1 promoters and HXT7 terminators. Through the gene editing technology, the CNE1 gene expression cassette is integrated to the I12 genome locus of Saccharomyces cerevisiae; the KAR2 gene expression cassette is integrated to the I4 and I32 genome loci of Saccharomyces cerevisiae; the PDI1 gene expression cassette is integrated to the I10 genome locus of Saccharomyces cerevisiae; the ERO1 gene expression cassette is integrated to the X3 genome locus of Saccharomyces cerevisiae; the IRE1 gene expression cassette is integrated to the I8 genome locus of Saccharomyces cerevisiae; the Hac1s gene expression cassette is integrated to the I28 genome locus of Saccharomyces cerevisiae; and the INO1 gene expression cassette is integrated to the I3 genome locus of Saccharomyces cerevisiae.

### Embodiment 10

In order to increase the intracellular solubility of cannabinoid, DAG acyltransferase polypeptide DGA1, G3P dehydrogenase polypeptide GPD1 and phosphatidic acid phosphatase polypeptide (PAH1) in the triacylglycerol synthesis pathway (TAG) are over-expressed. GPD1, DGA1 and PAH1 are respectively constructed to a TEF1 promoter and a TEF1 terminator. Through the gene editing technology, the GPD1, DGA1 and PAH1 gene expression cassettes are respectively integrated to the YORWΔ22, YCRWδ12 and YERCδ8 genome loci of Saccharomyces cerevisiae.

### Embodiment 11

A plurality of mutants H292L, H292R, Y417H, Y417R, N89Q-N499Q, R463C-D488C, T379S, K377R, N196Q, F171Y, S170T, R349K, F365Y, V138A, R532K, L524I, Y472F, N528Q and F353Y produced by site-directed mutagenesis of cannabichromenic acid synthase and a combination of double mutants, triple mutants and multiple mutants thereof are respectively constructed to GAL10 promoters and CYC1 terminators and respectively integrated to HIS3b and 511b genome loci through the gene editing technology.

### Embodiment 12

Production of Cannabichromenic Acid by Fermenting Recombinant Saccharomyces cerevisiae Strain

Recombinant Saccharomyces cerevisiae strains with high yield of cannabichromenic acid obtained in embodiment 2 are selected into test tubes containing 3-5 mL of YPD (10 g/L yeast extract, 20 g/L peptone and 20 g/L dextroglucose). Culture is carried out at 200 rpm at 30° C. for 24 h until glucose in the culture medium is depleted. Saturated strains are centrifuged at 4500 rpm and subcultured to a new culture medium containing 100 mL of YPG (10 g/L yeast extract, 20 g/L peptone and 20 g/L galactose), and culture is carried out at 200 rpm at 30° C. for 24-48 h. Fermentation liquor or high-pressure homogenization crushed bacteria liquid is centrifuged at 4500 rpm for 5 min, the supernatant is taken and extracted with an organic solvent, ethyl acetate, of ⅕ volume of supernatant (1 volume of ethyl acetate added to 5 volumes of fermentation liquor), and an organic layer is taken by centrifugation for rotary evaporation to obtain crude cannabichromenic acid which is heated at 105° C. for 15 min and then heated at 145° C. for 55 min, thus obtaining cannabichromene.

### Embodiment 13

Production of Cannabichromenic Acid by Fermenting Recombinant Yeast Strain

Recombinant Saccharomyces cerevisiae strains with high yield of cannabichromenic acid obtained in embodiment 5 are selected into test tubes containing 3-5 mL of YPD (10 g/L yeast extract, 20 g/L peptone and 20 g/L dextroglucose). Cells are cultured at 200 rpm at 30° C. for 24 h until glucose in the culture medium is depleted. Saturated strains are centrifuged at 4500 rpm and subcultured to a new culture medium containing 100 mL of YPG (10 g/L yeast extract, 20 g/L peptone, 20 g/L galactose, 1 mM olivetolic acid or 2 mM hexanoic acid), and culture is carried out at 200 rpm at 30° C. for 24-48 h. Fermentation liquor or high-pressure homogenization crushed bacteria liquid is centrifuged at 4500 rpm for 5 min, the supernatant is taken and extracted with an organic solvent, ethyl acetate, of ⅕ volume of supernatant (1 volume of ethyl acetate added to 5 volumes of fermentation liquor), and an organic layer is taken by centrifugation for rotary evaporation to obtain crude cannabichromenic acid which is heated at 105° C. for 15 min and then heated at 145° C. for 55 min, thus obtaining cannabichromene.

### Embodiment 14

Identification Method of Cannabichromenic Acid

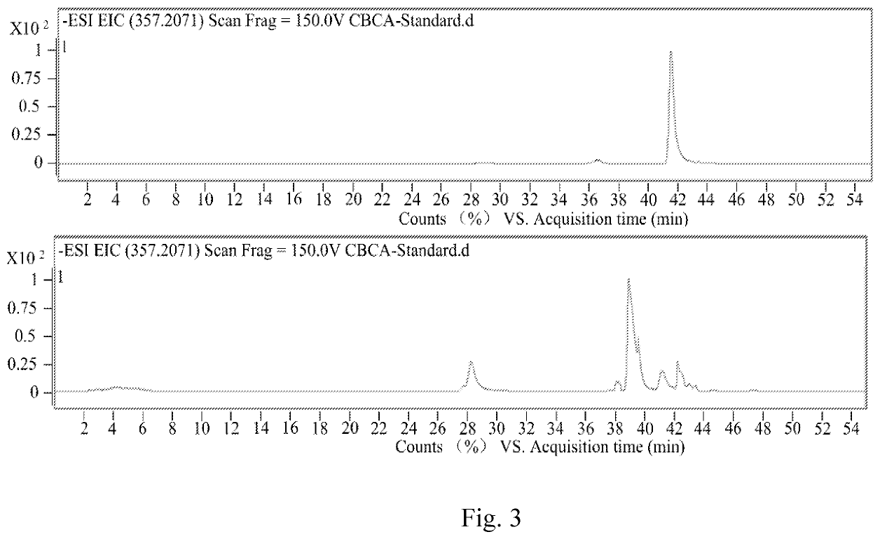

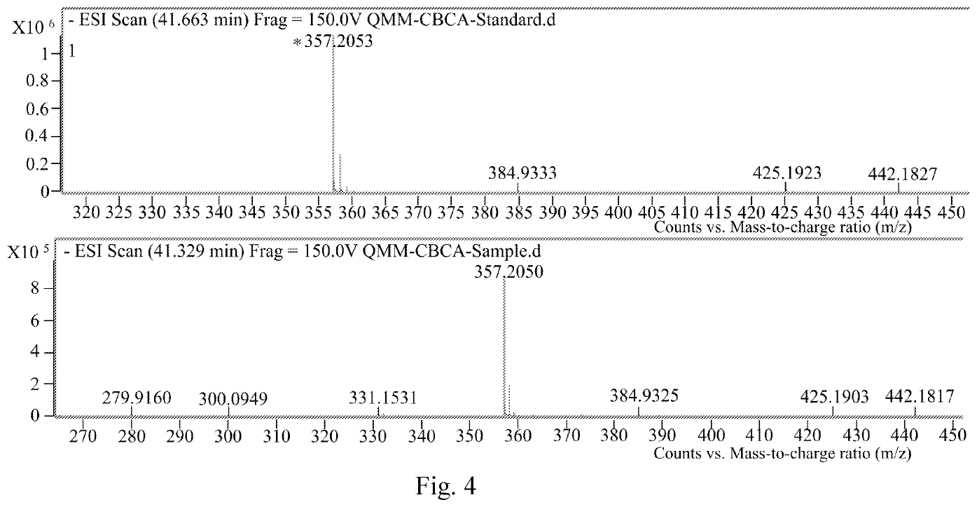

Fermentation liquor or high-pressure homogenization crushed bacteria liquid is centrifuged at 4500 rpm for 5 min, the supernatant is taken and extracted with an organic solvent, ethyl acetate, of ⅕ volume of supernatant (1 volume of ethyl acetate added to 5 volumes of fermentation liquor), an organic layer is taken by centrifugation for rotary evaporation, the evaporated substance is resuspended in a mixed solution of acetonitrile/0.05% aqueous formic acid solution (v/v of 80%/20%) and then filtered by an organic filter membrane to obtain high-concentration organic phase containing cannabigerolic acid, and detection and analysis are performed through a liquid chromatography/time-of-flight mass spectrometer. Results are shown in FIG. 3 and FIG. 4.

The related instruments, equipment and experimental parameters for detection and analysis of cannabichromenic acid are as follows: the instrument is Agilent 6224 TOF LC/MS, the column temperature is 25° C., the chromatographic column is Agilent C18 chromatographic column, the flow velocity is 0.2 mL/min, the mobile phase is aqueous solution (A) containing 0.05% formic acid and acetonitrile solution (B), and gradient elution conditions are: 0-40 min, 30%-98% acetonitrile; 40-50 min, 98% acetonitrile; and 50-51 min, 98%-30% acetonitrile, and the sampling volume is 20 μL.

Finally, it should be noted that the above embodiments are only used for describing the technical solution of the present invention rather than limiting the present invention. Although the present invention is described in detail by referring to the above embodiments, those ordinary skilled in the art should understand that: the technical solution recorded in each of the above embodiments can be still amended, or part or all of technical features therein can be replaced equivalently; and the amendments or replacements do not enable the essence of the corresponding technical solution to depart from the scope of the technical solution of various embodiments of the present invention.

1-18. (canceled)

19. A construction method of a recombinant Saccharomyces cerevisiae, wherein cannabigerolic acid synthase polypeptide and cannabichromenic acid synthase polypeptide are heterologously expressed by the recombinant Saccharomyces cerevisiae strain; p-ketothiolase polypeptide, 3-hydroxybutyryl coenzyme A dehydrogenase polypeptide, crotonase polypeptide, trans-2-enoyl-CoA reductase polypeptide, type III polyketide synthase polypeptide and olivetolic acid cyclase polypeptide are heterologously expressed by the recombinant Saccharomyces cerevisiae; acyl activating enzyme polypeptide and acetyl-CoA carboxylase polypeptide are heterologously expressed by the recombinant Saccharomyces cerevisiae;
wherein the construction method comprising the following steps:
1) respectively constructing expression cassettes of cannabigerolic acid synthase polypeptide and cannabichromenic acid synthase polypeptide, and inserting the above expression cassettes into a Saccharomyces cerevisiae genome through homologous recombination;
2) respectively constructing expression cassettes of p-ketothiolase polypeptide, 3-hydroxybutyryl coenzyme A dehydrogenase polypeptide, crotonase polypeptide, trans-2-enoyl-CoA reductase polypeptide, type III polyketide synthase polypeptide and olivetolic acid cyclase polypeptide, and inserting the above expression cassettes into the Saccharomyces cerevisiae genome obtained in step (1) through homologous recombination;
3) respectively constructing expression cassettes of acyl activating enzyme polypeptide and acetyl-CoA carboxylase polypeptide, and inserting the above expression cassettes into the Saccharomyces cerevisiae genome obtained in step (2) through homologous recombination;
4) respectively constructing expression cassettes of truncated HMG-CoA reductase polypeptide, acetoacetyl-CoA thiolase polypeptide, HMG-CoA synthase polypeptide, mutants of farnesyl pyrophosphate synthetase polypeptide, mevalonate kinase polypeptide ERG12, phosphomevalonate kinase polypeptide ERG8, mevalonate pyrophosphate decarboxylase polypeptide and isopentenyl-pyrophosphate delta isomerase polypeptide, and inserting the above expression cassettes into the Saccharomyces cerevisiae genome obtained in step (3) through homologous recombination;
5) respectively constructing expression cassettes of acetaldehyde dehydrogenase polypeptide, acetyl-CoA synthetase polypeptide and alcohol dehydrogenase polypeptide, and inserting the above expression cassettes into the Saccharomyces cerevisiae genome obtained in step (4) through homologous recombination.

20. The construction method of the recombinant Saccharomyces cerevisiae according to claim 19, comprising the following steps:
knocking out alcohol dehydrogenase polypeptide (ADH1) and YPL062W; and over-expressing pyruvate decarboxylase polypeptide PDC from Zea mays, endogenous acyl-CoA synthetase polypeptide FAA2, and acetyl-CoA synthetase polypeptide SeACS from Salmonella enterica.

21. A construction method of a recombinant Saccharomyces cerevisiae, wherein cannabigerolic acid synthase polypeptide and cannabichromenic acid synthase polypeptide are heterologously expressed by the recombinant Saccharomyces cerevisiae strain; p-ketothiolase polypeptide, 3-hydroxybutyryl coenzyme A dehydrogenase polypeptide, crotonase polypeptide, trans-2-enoyl-CoA reductase polypeptide, type III polyketide synthase polypeptide and olivetolic acid cyclase polypeptide are heterologously expressed by the recombinant Saccharomyces cerevisiae; acyl activating enzyme polypeptide and acetyl-CoA carboxylase polypeptide are heterologously expressed by the recombinant Saccharomyces cerevisiae;
wherein the construction method comprises the following steps:
(1) screening out promiscuous kinase polypeptide to be used from Salmonella enterica subsp and Saccharomyces cerevisiae; and screening out isopentenyl phosphate kinase polypeptide which can be successfully expressed in the Saccharomyces cerevisiae from multiple species of Thermoplasma acidophilum, Methanococcus vannielii, Methanolobus tindarius, Methanosalsum zhilinae, Methanococcus maripaludis, Methanococcoides burtonii and Arabidopsis thaliana;
(2) locating the promiscuous kinase polypeptide screened out, isopentenyl phosphokinase, acetoacetyl-CoA thiolase polypeptide, HMG-CoA synthase polypeptide, mevalonate kinase polypeptide ERG12, phosphomevalonate kinase polypeptide ERG8, mevalonate pyrophosphate decarboxylase polypeptide, isopentenyl-pyrophosphate delta isomerase polypeptide IDI, mutants of farnesyl pyrophosphate synthetase polypeptide ERG20mut, cannabigerolic acid synthase polypeptide CsPT4 and cannabichromenic acid synthase polypeptide CBCAS to peroxisomes;
(3) in order to increase the number and volume of the peroxisomes, knocking out peroxisomal membrane protein PEX31 and PEX32; and over-expressing peroxisomal membrane protein PEX3, PEX19, PEX11 and PEX34 so as to increase the production of cannabichromenic acid.

22. The construction method of the recombinant Saccharomyces cerevisiae according to claim 21, wherein:
in order to ensure the proper folding of CBCAS, a variety of molecular chaperones, foldases and transcription activators are screened simultaneously to improve the expression activity of CBCAS, including proteins involved in folding and endoplasmic reticulum quality control, cofactor FAD1p, UPR albumen IRE1*p, UPR activator Hac1s and endoplasmic reticulum size regulator INO1, wherein the proteins involved in folding and endoplasmic reticulum quality control comprise CNE1p, KAR2p, PDI1p and ERO1p.

23. The construction method of the recombinant Saccharomyces cerevisiae according to claim 21, wherein:
a plurality of mutants, respectively H292L, H292R, Y417H, Y417R, N89Q-N499Q, R463C-D488C, T379S, K377R, N196Q, F171Y, S170T, R349K, F365Y, V138A, R532K, L524I, Y472F, N528Q, F353Y and a combination of double mutants, triple mutants and multiple mutants, are produced by site-directed mutagenesis of cannabichromenic acid synthase, and the mutant with the best activity is screened out to produce cannabichromenic acid.

24. The construction method of the recombinant Saccharomyces cerevisiae according to claim 21, wherein:
in order to increase intracellular ATP and dissolved oxygen supply, the adenylate kinase polypeptide ADK1, phosphite dehydrogenase polypeptide ptxD from Pseudomonas stutzeri and Vibrio hyaluronicus hemoglobin polypeptide VHB are over-expressed to increase the synthesis rate of ATP, so as to promote cell growth and cannabinoid production.

25. The construction method of the recombinant Saccharomyces cerevisiae according to claim 21, wherein:
cannabichromenic acid synthase polypeptide is located to the lipid droplets in order to obtain higher reaction rate and higher productivity by increasing the local concentration of substrate and enzyme, meanwhile, in order to improve the solubility of cannabinoid, the key enzyme polypeptides of DAG acyltransferase polypeptide DGA1, G3P dehydrogenase polypeptide GPD1 and phosphatidic acid phosphatase polypeptide PAH1 in the triacylglycerol pathway TAG are over-expressed, and the key protein polypeptide SEI1 for lipid droplet synthesis is knocked out to increase lipid level and lipid droplet aggregation; and cannabichromenic acid synthase polypeptide is located to the lipid droplets in order to obtain higher reaction rate and higher productivity and expand the storage capacity of engineered yeast to synthesize cannabinoids by increasing the local concentration of substrate and enzyme.

26. The construction method of the recombinant Saccharomyces cerevisiae according to claim 21, wherein:
the key enzyme polypeptides in the metabolic pathway of cannabichromene are simultaneously integrated to the rDNA locus of the Saccharomyces cerevisiae to achieve multi-copy expression of a plurality of key enzyme polypeptides.

27. The construction method of the recombinant Saccharomyces cerevisiae according to claim 21, wherein:
Gal80 is knocked out to remove the inhibitory effect thereof on Gal4, and meanwhile, the promoter of Gal4 is replaced to remove the inhibitory effect of glucose; and galactose is no longer needed as an inducer, and the yield of cannabichromenic acid CBCA is optimized through mixed fermentation of glucose and ethanol.

28. A method for producing cannabichromenic acid by fermenting recombinant Saccharomyces cerevisiae, wherein cannabigerolic acid synthase polypeptide and cannabichromenic acid synthase polypeptide are heterologously expressed by the recombinant Saccharomyces cerevisiae strain; β-ketothiolase polypeptide, 3-hydroxybutyryl coenzyme A dehydrogenase polypeptide, crotonase polypeptide, trans-2-enoyl-CoA reductase polypeptide, type III polyketide synthase polypeptide and olivetolic acid cyclase polypeptide are heterologously expressed by the recombinant Saccharomyces cerevisiae; acyl activating enzyme polypeptide and acetyl-CoA carboxylase polypeptide are heterologously expressed by the recombinant Saccharomyces cerevisiae;
wherein the method comprising the following steps:
1) culturing the recombinant Saccharomyces cerevisiae of claim 19 in an appropriate culture medium for a period of time;
2) recycling cannabichromenic acid generated by fermentation;
3) decarboxylating cannabichromenic acid by heating or storage to form cannabichromene.

29. The method according to claim 28, wherein the culture medium contains one or a mixture of more of glucose, galactose, glycerine, ethanol, starch, hexanoic acid and olivetolic acid.

30. The method according to claim 28, wherein the culture conditions are as follows: the revolving speed is 50-300 rpm, the temperature is 28-32° C., and the culture time is 24-120 h.

31. The method according to claim 30, wherein the process of recycling cannabichromenic acid generated by fermentation comprises the step of extracting cannabichromenic acid from fermentation liquor or cell crushing liquid with an organic solvent.

32. The method according to claim 31, wherein the organic solvent is one or a mixture of more of ethyl acetate, hexane, heptane, petroleum ether and chloroform.

33. The method according to claim 32, wherein the cell crushing liquid is obtained by crushing host cells through high-pressure homogenization crushing, ultrasonic crushing, ball-milling brushing, repeated freezing and thawing crushing or enzymatic solubilization crushing.

34. The method according to claim 33, wherein the cell crushing liquid is obtained by crushing host cells through high-pressure homogenization crushing, ultrasonic crushing, ball-milling brushing, repeated freezing and thawing crushing or enzymatic solubilization crushing.

35. The method according to claim 29, wherein the culture conditions are as follows: the revolving speed is 50-300 rpm, the temperature is 28-32° C., and the culture time is 24-120 h.
